SEQUENCE LISTING (13 sequences)

SEQ ID NO: 1 - DNA, 2173 bp, Homo sapiens
  CDS (66)...(878)
cccttcccgggccggcgggctccccgggctccccgccgccgccccgtgcgccccgggagcccggcatgctgcgccacgtgctgctcgccgcgctctgcctggcggcgtccccagcgcccgcgcgcgcctgccagctgcactccgagtggaggcccctgagcgagggctgccgcgcggagctggccgagaccatcgtgtacgccagggtgctgccgctgcaccccgaggcgcccggcctctacaaccacctgccctggcagtaccacgccggccaagggggcctcttctactcggccgaggtcgagatgctgtgcgaccaggcgtggggcagcatgctggaggtgccggccggctccaggctcaacctcaccggcctgggctacttctcgtgccactcccacaccgtggtccaggactactcctatttcttcttcctcaggatggatgaaaattataacctcttgcctcacggagtcaatttccgagatgccatcttcccagacactcaagagaacagaaggatgttttctagccttttccagttttcaaactgttcgcaagggcagcagctggcgactttctccagtgactgggaaatccaggaagacagtaggctcatgtgctcctcggtgcagaaggccttgtttgaggaggaggaccacgtcaagaaactgcagcagaaagtggccaccctggagaagcgcaaccggcagctccgggagcgagtgaagaaggtcaagaggtccttgcggcaggcgcgtaagaagggccgccacctggagctggcgaaccagaaactcagtgagaagctggcggcgggcgcgctgccgcacatcaacgcccgggggcccgtgcgccccccctacctgcgggggtaacgggcctgggggctgccaggtgtgcagggccaatcctggcggtaattgagaatgagtgaggtttcgtacatgcagctatttcaagggttgtaagagtttttgtttttaatcacgcatttggtagagtctaaatggataaaatgcaaggcttgctttccccttgggtgctggcctcaatgtcagaccccacgcgctgccccttcctggcctgaccccagacgcagtgcctggcagtccagaggcagtgggatccctgagtgctgaatgctcgcctgcagagcagcccagaaagagccctgactggggagagaacattttagaatctctagtgtaaaagacatcaacgtgcttagcctttatttcagaaaaaaatcagggtggtttccagctccccagtccaggacaaccattagtcctgatgagtgagctgacgctggtgctggaacctgctggcacctcactggccacatctttggaaggggatggtggccttgcatccaagatgcctgaaaatcagcacgtgcagggcctccctatccagccagcattttccttccagctgaggcaggtgaagacttcataagctcatcacaggggagggaattaggagcagggcagcaggtaattaaacaagataaattatacctgatttccaacaccagctacaaagagttgaagatgatacctatgggtcgcgttaacacagggggcaactgccttgatcggcctgccatgggtcatcagactgcttcctaaattgagagaaactgagcaatctctcagccactgctatagtctaacttcttgtttgctgagtaattgtttctaatgtctctgaactcaaagtgaggtgctccaagacgctgtgaacttctgcaaagacacctccttacctactgggatcacgtgacctgacctcactcccagccaggctcccaaagggctcattccagccattccaatctcttcttctttatgcaaacacttttcccccacaacaagccttgtttgttccgataggaatacgtgtacgtcagtgcacttgtccttacgtcagttccttacaccaccaaagcacttcacctttctggaaataaaacttttaagacactactataagtaaaaatgagagtattcactagacttattgctcaggcacatttgagtgggtcccagctgtgtgattaagaagtcaactgggtggccttttctgggttatcttctgatcatggcctttcaacccaacaagggcccttccctgctcttccacataaaggtcttggctttatagaact

SEQ ID NO: 2 - protein, 270 aa, Homo sapiens
MLRHVLLAALCLAASPAPARACQLHSEWRPLSEGCRAELAETIVYARVLPLHPEAPGLYNHLPWQYHAGQGGLFYSAEVEMLCDQAWGSMLEVPAGSRLNLTGLGYFSCHSHTVVQDYSYFFFLRMDENYNLLPHGVNFRDAIFPDTQENRRMFSSLFQFSNCSQGQQLATFSSDWEIQEDSRLMCSSVQKALFEEEDHVKKLQQKVATLEKRNRQLRERVKKVKRSLRQARKKGRHLELANQKLSEKLAAGALPHINARGPVRPPYLRG

SEQ ID NO: 3 - DNA, 813 bp, Homo sapiens
  CDS (1)...(813)
atgctgcgccacgtgctgctcgccgcgctctgcctggcggcgtccccagcgcccgcgcgcgcctgccagctgcactccgagtggaggcccctgagcgagggctgccgcgcggagctggccgagaccatcgtgtacgccagggtgctgccgctgcaccccgaggcgcccggcctctacaaccacctgccctggcagtaccacgccggccaagggggcctcttctactcggccgaggtcgagatgctgtgcgaccaggcgtggggcagcatgctggaggtgccggccggctccaggctcaacctcaccggcctgggctacttctcgtgccactcccacaccgtggtccaggactactcctatttcttcttcctcaggatggatgaaaattataacctcttgcctcacggagtcaatttccgagatgccatcttcccagacactcaagagaacagaaggatgttttctagccttttccagttttcaaactgttcgcaagggcagcagctggcgactttctccagtgactgggaaatccaggaagacagtaggctcatgtgctcctcggtgcagaaggccttgtttgaggaggaggaccacgtcaagaaactgcagcagaaagtggccaccctggagaagcgcaaccggcagctccgggagcgagtgaagaaggtcaagaggtccttgcggcaggcgcgtaagaagggccgccacctggagctggcgaaccagaaactcagtgagaagctggcggcgggcgcgctgccgcacatcaacgcccgggggcccgtgcgccccccctacctgcgggggtaa

SEQ ID NO: 4 - protein, 22 aa, Homo sapiens
MLRHVLLAALCLAASPAPARAC

SEQ ID NO: 5 - protein, 248 aa, Homo sapiens
QLHSEWRPLSEGCRAELAETIVYARVLPLHPEAPGLYNHLPWQYHAGQGGLFYSAEVEMLCDQAWGSMLEVPAGSRLNLTGLGYFSCHSHTVVQDYSYFFFLRMDENYNLLPHGVNFRDAIFPDTQENRRMFSSLFQFSNCSQGQQLATFSSDWEIQEDSRLMCSSVQKALFEEEDHVKKLQQKVATLEKRNRQLRERVKKVKRSLRQARKKGRHLELANQKLSEKLAAGALPHINARGPVRPPYLRG

SEQ ID NO: 6 - DNA, 2895 bp, Mus musculus
  CDS (399)...(1220)
cgtctttgtttcgttttctgttctgcgccgttacagatccaagctctgaaaaaccagaaagttaactggtaagtttagtctttttgtcttttatttcaggtcccggatccggtggtggtgcaaatcaaagaacttgctcctcagtggatgttgcctttacttctaggcctgtacggaagtgttacttctgctctaaaagctgcggaattctaatacgactcactatagggagtcgacccacgcgtccggctagccgtgcacccagctctccggagcgcgtgcaggcgagccgagcgccccgtccgcggttctcgggcaggcgctgcgggctccccggctccccgccgtcccgggcacccgggcgggccatgcgcccgggctagagcgtagccgccggcatgccgctcccgctgctgctcgccgcgctctgcctcgccgcctccccggcgcccgcgcgcgcctgccagctgccgtcggagtggagacccttgagcgaaggctgccgcgccgagctagccgagaccatcgtgtatgccaaggtgctggcgctgcaccccgaggtgcctggcctctacaactacctgccgtggcagtaccaagctggagagggagggctcttctactccgccgaggtggagatgctgtgtgaccaggcgtggggcagtatgttggaggtgcccgccggctcccggctcaaccttactggtctgggctacttctcctgccactcccacacggtggtccaggactactcttatttcttctttgtaaggatggatgagaattacaatctcttgccacacggagtcaatttccaagatgccatctttccagacacccaggagaacagaaggatgttttccagccttttccagtttgctaactgttcccaagggcagcagctgacgactttctcgagtgactgggaggtccaggaagacaacaggctcatgtgctcctcggtgcagaaggccctgtttgaggaagaggaccatgtcaagaagctgcagcagaaggtggccaccctggagaagcggaacaggcagctccgagagcgggtcaagaaggtcaagaggtctctgaggcaggcacggaagaacagccgccacctggagctggtgaaccaaaaactcaatgagaagctaggggcctccagtgctcagcagcacattaacgctctgggccgggagcccgtgcgtgccccctatctgcatgggtagcaaggggcaggtgggatgctggcctgcctttgccttgtgccggctgactgagaacaaccgcaagctctatatatgcacttacttcaaacattatagtagctatccatttggcatttggtagagtctaactgggtaaattgtcaggttttgctttctttcccattgggtcctggccttactggccatttgacttcagatcccatgtgccacatgtcctctttctgtcctggtgccacataagagccttaaaaccggaaggcaatagtatcttgagtctggccctcagcctctagagagttccttcgctggggggagaacattctagaacctcggtatacatgaagatatcacaggactctcttgtttcagaagggagtcggtgttgaggctcagcaaccagggttggcacacgcatgcgtgttctaaaatgttctgaatgttcgtgccttttctcgtgtttttagaaatggtgtctacgtgtaatgtgcagggcacccacccgtgcttcagtcaccatttctcccaaagctgaaagatgttaagacttaatgagttcattacaggtaagggaactgggcgaggagctgaggcagctagtaattggtaaagataaattatatctgttttccaaccccagctccttggggctgactgattcattccgattttaatctaagcgaggttgcttttatagacataccatattcatcactcatgtttcaaactggattaagctcaacctaactttgtggagtttaactttgtgttagcgcagattgcatggctccgagtctaaggtcactgacttgcacacactgagactttctactctgcagaaacagctccctgcctgaaggggttcatagctctatgctagggccaagggctgtcaggattaacccaagctactatgaacctccaagcctctgacccaacacacacacacacacacacacacacacacacacacacactgctcaggttctcataaaaggtctacgtgtaccccatagaagtatttgcataacaaacacttcttttaataagagtgctttcactatccttcccatggccatgtttgattatgtcccatttgtgttactaagaagtcaatggagtggctgctggttcatcgcctctaatggtggccctccagatctaatgccgtgccacactgttccaccaatagaaagaatccttcagctctacaccattgaccacctagaacccattccagacacatgatactgataaaagtggtcattactgtcacactgccgcctttctgcttgcaggaagtccaggcctctgtgagaagtccctcctgtcacatgagtattggtaggggtggggataagtttgtgcccttggctggtcagggcatgatgggaaggaacttctattctgggaccaatcttcatccactctcccccactcagcctctttaagcagtgctattcataatatgctacagaggtgtgccttctcattaattctccctgggagcattaagttaaagcgcttttgttacctcccaagggaatgaatggcaagcttaggggtgatggagtcaggactcggagaactgtggaagccgaagatgtcttatgttctgcttttccaataaacatgagcagtgcttccaatggctgtggaaaaaaaaaaaaaaaaaaaa

SEQ ID NO: 7 - protein, 273 aa, Mus musculus
MPLPLLLAALCLAASPAPARACQLPSEWRPLSEGCRAELAETIVYAKVLALHPEVPGLYNYLPWQYQAGEGGLFYSAEVEMLCDQAWGSMLEVPAGSRLNLTGLGYFSCHSHTVVQDYSYFFFVRMDENYNLLPHGVNFQDAIFPDTQENRRMFSSLFQFANCSQGQQLTTFSSDWEVQEDNRLMCSSVQKALFEEEDHVKKLQQKVATLEKRNRQLRERVKKVKRSLRQARKNSRHLELVNQKLNEKLGASSAQQHINALGREPVRAPYLHG

SEQ ID NO: 8 - DNA, 822 bp, Mus musculus
  CDS (1)...(822)
atgccgctcccgctgctgctcgccgcgctctgcctcgccgcctccccggcgcccgcgcgcgcctgccagctgccgtcggagtggagacccttgagcgaaggctgccgcgccgagctagccgagaccatcgtgtatgccaaggtgctggcgctgcaccccgaggtgcctggcctctacaactacctgccgtggcagtaccaagctggagagggagggctcttctactccgccgaggtggagatgctgtgtgaccaggcgtggggcagtatgttggaggtgcccgccggctcccggctcaaccttactggtctgggctacttctcctgccactcccacacggtggtccaggactactcttatttcttctttgtaaggatggatgagaattacaatctcttgccacacggagtcaatttccaagatgccatctttccagacacccaggagaacagaaggatgttttccagccttttccagtttgctaactgttcccaagggcagcagctgacgactttctcgagtgactgggaggtccaggaagacaacaggctcatgtgctcctcggtgcagaaggccctgtttgaggaagaggaccatgtcaagaagctgcagcagaaggtggccaccctggagaagcggaacaggcagctccgagagcgggtcaagaaggtcaagaggtctctgaggcaggcacggaagaacagccgccacctggagctggtgaaccaaaaactcaatgagaagctaggggcctccagtgctcagcagcacattaacgctctgggccgggagcccgtgcgtgccccctatctgcatgggtag

SEQ ID NO: 9 - protein, 23 aa, Mus musculus
MPLPLLLAALCLAASPAPARACQ

SEQ ID NO: 10 - protein, 251 aa, Mus musculus
QLPSEWRPLSEGCRAELAETIVYAKVLALHPEVPGLYNYLPWQYQAGEGGLFYSAEVEMLCDQAWGSMLEVPAGSRLNLTGLGYFSCHSHTVVQDYSYFFFVRMDENYNLLPHGVNFQDAIFPDTQENRRMFSSLFQFANCSQGQQLTTFSSDWEVQEDNRLMCSSVQKALFEEEDHVKKLQQKVATLEKRNRQLRERVKKVKRSLRQARKNSRHLELVNQKLNEKLGASSAQQHINALGREPVRAPYLHG

SEQ ID NO: 11 - DNA, 20 bp, Artificial Sequence
  FARS1 primer
aatggtggccctccagatct

SEQ ID NO: 12 - DNA, 23 bp, Artificial Sequence
  FARS1 primer
ggtcaatggtgtagagctgaagg

SEQ ID NO: 13 - DNA, 28 bp, Artificial Sequence
  FARS1 probe
ccgtgccacactgttccaccaatagaaa

(19) United States
(12) Patent Application Publication
(10) Pub. No.: US 2003/0113889 A1
(43) Pub. Date: Jun. 19, 2003

(54) FARS1, a human secreted protein and uses thereof

(75) Inventors: Ruth E. Gimeno, Wellesley, MA (US); Mehran M. Khodadoust, Brookline, MA (US)

Correspondence Address: Jean M. Silveri, MILLENNIUM PHARMACEUTICALS, INC., 75 Sidney Street, Cambridge, MA, 02139, US

(73) Assignee: Millennium Pharmaceuticals, Inc.

(21) Appl. No.: 10/152,874

(22) Filed: May 21, 2002

Related U.S. Application Data
(60) Provisional application No. 60/292,471, filed on May 21, 2001.

(51) Int. Cl. C12N 9/00; C07H 21/04; C12P 21/02; C12N 5/06

(52) U.S. Cl. 435/183; 435/69.1; 435/320.1; 435/325; 536/23.2

(57) ABSTRACT

The invention provides isolated nucleic acids molecules, designated FARS1 nucleic acid molecules, which encode novel secreted proteins. The invention also provides antisense nucleic acid molecules, recombinant expression vectors containing FARS1 nucleic acid molecules, host cells into which the expression vectors have been introduced, and nonhuman transgenic animals in which a FARS1 gene has been introduced or disrupted. The invention still further provides isolated FARS1 proteins, fusion proteins, antigenic peptides and anti-FARS1 antibodies. Diagnostic and therapeutic methods utilizing compositions of the invention are also provided.

### CROSS-REFERENCE TO RELATED APPLICATION

This application claims the benefit of U.S. Provisional Application No. 60/292,471, filed May 21, 2001, the contents of which are incorporated herein by this reference.

### BACKGROUND OF THE INVENTION

Secreted proteins play an important role in energy homeostasis by transmitting signals between different tissues as well as by signaling within tissues. One example of a secreted protein involved in energy metabolism is leptin, a protein secreted by adipocytes that acts as a sensor of body energy stores and transmits this information to the hypothalamus (see Friedman and Halaas, 1998, Nature:395:763-70, for review). The dramatic obesity caused by deletion of leptin has focused attention on the importance of proteins secreted by adipocytes for energy homeostasis. Recent research has identified a number of adipocyte secreted proteins, including, but not limited to, TNF-alpha (Hotamisligil et al. (1993) Science 259:87-91), adipsin (Flier et al.(1987) Science 237:405-408), adipoQ (Hu et al. (1996) J. Biol. Chem. 271:10697-10703), FIAF (Kersten et al. (2000) J. Biol. Chem. 275:28488-28493), and PGAR (Yoon et al.(2000) Mol. Cell Biol. 20:5343-9). While some of these proteins, such as adipoQ and leptin, are specifically expressed in adipocytes, or are highest in adipose tissue (e.g., FIAF), others, such as TNF-alpha, are also secreted by other tissues. TNF-alpha has been suggested to have an important role in regulating insulin signalling and glucose metabolism in fat and muscle. AdipoQ has recently been shown to be important in controlling nutrient use in muscle and liver (Fruebis et al. (2001) Proc. Natl. Acad. Sci. USA 98:2005-2010). The function of adipsin, FIAF and PGAR is less clear. However, regulation of expression of these proteins in genetic models of obesity (adipsin, AdipoQ, PGAR), by starvation (FIAF) or by PPARgamma agonists (FIAF, PGAR) suggests that these proteins play a role in energy homeostasis, lipid metabolism and/or diabetes.

### SUMMARY OF THE INVENTION

The present invention is based, in part, on the discovery of a novel secreted protein, referred to herein as “FARS1,” which is expressed at high levels in adipocytes and which increased expression is associated with overfeeding and decreased expression is associated with underfeeding. The nucleotide sequence of a cDNA encoding human FARS1 is shown in SEQ ID NO:1, and the nucleotide sequence of the coding region is depicted in SEQ ID NO:3. The amino acid sequence encoded by the coding region of SEQ ID NO:1 or SEQ ID NO:3 is shown in SEQ ID NO:2. The signal peptide and the sequence of the mature protein of the amino acid sequence shown in SEQ ID NO:2 are depicted in SEQ ID NO:4 and SEQ ID NO:5, respectively. The nucleotide sequence of a cDNA encoding murine FARS1 is shown in SEQ ID NO:6, and the nucleotide sequence of the coding region is depicted in SEQ ID NO:8. The amino acid sequence encoded by the coding region of SEQ ID NO:6 or SEQ ID NO:8 is shown in SEQ ID NO:7. The signal peptide and the sequence of the mature protein of the amino acid sequence shown in SEQ ID NO:7 are depicted in SEQ ID NO:9 and SEQ ID NO:10, respectively.

Accordingly, in one aspect the invention features an isolated nucleic acid molecule which is at least 80% identical to the nucleotide sequence of SEQ ID NO:1, SEQ ID NO:3, SEQ ID NO:6, SEQ ID NO:8, or having the sequence of the DNA insert of the plasmid deposited with the ATCC as Accession Number ______. In another aspect, the invention features an isolated nucleic acid molecule which is a biologically active fragment of the nucleotide sequence of SEQ ID NO:1, SEQ ID NO:3, SEQ ID NO:6, SEQ ID NO:8, or having the sequence of the DNA insert of the plasmid deposited with the ATCC as Accession Number ______. In another aspect, the invention features an isolated nucleic acid molecule which encodes a polypeptide comprising the amino acid sequence of SEQ ID NO:2, SEQ ID NO:5, SEQ ID NO:7, SEQ ID NO:10, or the amino acid sequence encoded by the cDNA insert of the plasmid deposited with the ATCC as Accession Number ______, or a biologically active fragment thereof. In another aspect, the invention features a nucleic acid molecule which encodes a naturally occurring allelic variant of a polypeptide comprising the amino acid sequence of SEQ ID NO:2, SEQ ID NO:5, SEQ ID NO:7, OR SEQ ID NO:10, or the amino acid sequence encoded by the cDNA insert of the plasmid deposited with the ATCC as Accession Number ______. In other embodiments, the invention provides a nucleic acid molecule which encodes a naturally occurring allelic variant of a polypeptide comprising the amino acid sequence of SEQ ID NO:2, SEQ ID NO:5, SEQ ID NO:7, OR SEQ ID NO:10, or the amino acid sequence encoded by the cDNA insert of the plasmid deposited with the ATCC as Accession Number ______, wherein the nucleic acid molecule hybridizes to a nucleic acid molecule comprising SEQ ID NO:1, SEQ ID NO:3, SEQ ID NO:6, SEQ ID NO:8, or a complement thereof, under stringent conditions.

In other embodiments, the invention provides isolated FARS1 nucleic acid molecules having the nucleotide sequence shown in SEQ ID NO:1, SEQ ID NO:3, SEQ ID NO:6, SEQ ID NO:8, or the sequence of the DNA insert of the plasmid deposited with ATCC Accession Number ______. In a preferred embodiment, the isolated nucleic acid molecule encodes a polypeptide having the amino acid sequence of SEQ ID NO:2, SEQ ID NO:5, SEQ ID NO:7, or SEQ ID NO:10. In another preferred embodiment, the invention provides nucleic acid molecules that are substantially identical (e.g., naturally occurring allelic variants) to the nucleotide sequence shown in SEQ ID NO:1, SEQ ID NO:3, SEQ ID NO:6, SEQ ID NO:8, or the sequence of the DNA insert of the plasmid deposited with ATCC Accession Number ______. In still other embodiments, the invention provides a nucleic acid molecule which hybridizes under stringent hybridization conditions to a nucleic acid molecule comprising the nucleotide sequence of SEQ ID NO:1, SEQ ID NO:3, SEQ ID NO:6, SEQ ID NO:8, or the sequence of the DNA insert of the plasmid deposited with ATCC Accession Number ______.

In a related aspect, the invention further provides nucleic acid constructs which include a FARS1 nucleic acid molecule. In certain embodiments, the nucleic acid molecules of the invention are operatively linked to native or heterologous regulatory sequences. Also included are vectors and host cells containing the FARS1 nucleic acid molecules of the invention, e.g., vectors and host cells suitable for producing FARS1 nucleic acid molecules and polypeptides.

In another aspect, the invention features, FARS1 polypeptides, and biologically active or antigenic fragments thereof, that are useful, e.g., as reagents or targets in assays applicable to treatment and diagnosis of FARS1-mediated or -related disorders. In another embodiment, the invention provides FARS1 polypeptides having a FARS1 activity. Preferred polypeptides are FARS1 proteins that contain six cysteines and six dibasic cleavage sites, as described herein, and preferably, have a FARS1 activity, e.g., a FARS1 activity as described herein. In other embodiments, the invention provides FARS1 polypeptides, e.g., a polypeptide which is encoded by a nucleic acid molecule comprising a nucleotide sequence which is at least 80% identical to a nucleic acid comprising the nucleotide sequence of SEQ ID NO:1, SEQ ID NO:3, SEQ ID NO:6, SEQ ID NO:8, or the amino acid sequence encoded by the DNA insert of the plasmid deposited with the ATCC as Accession Number ______; a polypeptide having an amino acid sequence which is at least 80% identical to the amino acid sequence shown in SEQ ID NO:2, SEQ ID NO:5, SEQ ID NO:7, SEQ ID NO:10, or the amino acid sequence encoded by the cDNA insert of the plasmid deposited with ATCC Accession Number ______; an amino acid sequence that is substantially identical (e.g., a naturally occurring variant) to the amino acid sequence shown in SEQ ID NO:2, SEQ ID NO:5, SEQ ID NO:7, SEQ ID NO:10, or the amino acid sequence encoded by the cDNA insert of the plasmid deposited with the ATCC as Accession Number ______; an amino acid sequence encoded by a nucleic acid molecule having a nucleotide sequence which hybridizes under stringent hybridization conditions to a complement of a nucleic acid molecule comprising the nucleotide sequence of SEQ ID NO:1, SEQ ID NO:3, SEQ ID NO:6, SEQ ID NO:8, or the sequence of the DNA insert of the plasmid deposited with ATCC Accession Number ______, wherein the nucleic acid encodes a full length FARS1 protein, or a biologically active fragment thereof.

In a related aspect, the invention provides FARS1 polypeptides or fragments operatively linked to non-FARS1 polypeptides to form fusion proteins.

In another aspect, the invention features antibodies, and antigen-binding fragments thereof, that react with, or more preferably specifically or selectively bind, FARS1 polypeptides.

In another aspect, the invention features methods for producing a FARS1 polypeptide, comprising culturing a host cell which contains a nucleic acid molecule encoding the naturally occurring variant under conditions in which the nucleic acid molecule is expressed, and wherein the nucleic acid molecule encodes the amino acid sequence of SEQ ID NO:2, SEQ ID NO:5, SEQ ID NO:7, SEQ ID NO:1,0, the amino acid sequence encoded by the DNA insert of the plasmid deposited with the ATCC as Accession Number ______, or a biologically active fragment thereof. In another embodiment, the FARS1 polypeptide is a naturally occurring variant of the FARS1 polypeptide which is encoded by a nucleic acid molecule which hybridizes under stringent conditions to a complement of a nucleic acid molecule comprising SEQ ID NO:1, SEQ ID NO:3, SEQ ID NO:6, or SEQ ID NO:8.

The invention also provides assays for determining the activity of or the presence or absence of FARS1 polypeptides or nucleic acid molecules in a sample, e.g., a biological sample, including for disease diagnosis.

In a related aspect, the invention provides kits for detecting the activity of or the presence or absence of FARS1 polypeptides or nucleic acid molecules in a sample, e.g., a biological sample, including for disease diagnosis.

In another aspect, the invention provides methods of screening for compounds that modulate the expression or activity of the FARS1 polypeptides or nucleic acids. In still another aspect, the invention provides a process for modulating FARS1 nucleic acid expression or polypeptide activity, e.g. using the screened compounds. In certain embodiments, the methods involve treatment of conditions related to aberrant activity or expression of the FARS1 polypeptides or nucleic acids, such as conditions involving aberrant or deficient lipid and cholesterol metabolism, obesity, diabetes, and cellular proliferation or differentiation.

In a further aspect the invention provides assays for determining the presence or absence of a genetic alteration in a FARS1 polypeptide or nucleic acid molecule, including using such assays for disease diagnosis.

Other features and advantages of the invention will be apparent from the following detailed description, and from the claims.

### DETAILED DESCRIPTION

The human FARS1 sequence (shown in SEQ ID NO:1), which is approximately 2173 nucleotides long including untranslated regions, contains a predicted methionine-initiated coding sequence of about 813 nucleotides, starting at nucleotide residue 66 of SEQ ID NO:1 and including the termination codon at nucleotide residue 878. The coding sequence encodes a 270 amino acid protein (shown in SEQ ID NO:2). The human FARS1 protein of SEQ ID NO:2 is predicted to include an amino-terminal hydrophobic amino acid sequence, consistent with a signal sequence, of about 21 amino acids (from amino acid 1 to about amino acid 21 of SEQ ID NO:2 and the sequence shown in SEQ ID NO:4; PSORT, Nakai, K., and Kanehisa, M. (1992) Genomics 14:897-911), which upon cleavage results in the production of a mature protein form. This mature protein form is approximately 249 amino acid residues in length (from about amino acid 22 to amino acid 270 of SEQ ID NO:2 and the sequence shown in SEQ ID NO:5). Additionally, there are seven cysteine residues at about amino acids 11 and 22, which are in the predicted signal sequence (SEQ ID NO:4), and at about amino acids 35, 83, 109, 163, and 186 of SEQ ID NO:2, which are found in the mature protein sequence at positions 13, 61, 87, 141, and 164 of SEQ ID NO:5, and six dibasic cleavage sites at about amino acids R151-R152, K201-K202, K212-K213, K222-K223, K225-R226, and R232-K233 of SEQ ID NO:2. The presence of such cleavage sites indicates potential proteolytic processing of the FARS1 protein by proteases (Steiner, (1998) Curr. Opin. Chem. Biol. 2:31-39).

To determine whether a polypeptide or protein of interest has a conserved sequence or domain common to members of a protein family, the amino acid sequence of the protein can be searched against a database of profile hidden Markov models (profile HMMs), which uses statistical descriptions of a sequence family's consensus (e.g., HMMER, version 2.1.1) and PFAM, a collection of multiple sequence alignments and hidden Markov models covering many common protein domains (e.g., PFAM, version 5.5) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the PFAM database can be found in Sonhammer et al., (1997) Proteins 28(3):405-420 and a detailed description of HMMs can be found, for example, in Gribskov et al., (1990) Meth. Enzymol. 183:146-159; Gribskov et al., (1987) Proc. Natl. Acad. Sci. USA 84:4355-4358; Krogh et al., (1994) J. Mol. Biol. 235:1501-1531; and Stultz et al., (1993) Protein Sci. 2:305-314, the contents of which are incorporated herein by reference. See also, for example, The HMMER User's Guide at http://hmmer.wustl.edu/hmmer-html. For general information regarding PFAM identifiers, PS prefix and PF prefix domain identification numbers, refer to Sonnhammer et al. (1997) Protein 28:405-420 and http//www.psc.edu/general/software/packages/pfam/pfam.html. See also, for example, http://www.expasy.ch/prosite and http://smart.embl-heidelberg.de/.

Using such search tools, e.g., Prosite (version 12.2), the human FARS1 polypeptide was found to contain the following structural characteristics:

(i) two N-glycosylation sites (Prosite Accession Number PS00001) at about amino acids 100 to 103 and 162 to 165 of SEQ ID NO:2;

(ii) two protein kinase C phosphorylation sites (Prosite Accession Number PS00005) at about amino acids 227 to 229 and 246 to 248 of SEQ ID NO:2 predicted by Prosite pattern match (Prosite version 12.2);

(iii) two casein kinase II phosphorylation sites (Prosite Accession Number PS00006) at about amino acids 89 to 92 and 174 to 177 predicted by Prosite pattern match (Prosite version 12.2); and

(iv) three N-myristoylation sites (Prosite Accession Number PS00008) at about amino acids 72 to 77, 96 to 101, and 166 to 171 predicted by Prosite pattern match (Prosite version 12.2).

A plasmid containing the nucleotide sequence encoding human FARS1 was deposited with American Type Culture Collection (ATCC), 10801 University Boulevard, Manassas, Va. 20110-2209, on ______ and assigned Accession Number ______.

The murine FARS1 sequence (shown in SEQ ID NO:6), which is approximately 2895 nucleotides long including untranslated regions, contains a predicted methionine-initiated coding sequence of about 822 nucleotides, including the termination codon (nucleotides indicated as coding in SEQ ID NO:6 and the sequence shown in SEQ ID NO:8). The coding sequence encodes a 273 amino acid protein (shown in SEQ ID NO:7). The murine FARS1 protein of SEQ ID NO:7 is predicted to include an amino-terminal hydrophobic amino acid sequence, consistent with a signal sequence, of about 21 amino acids (from amino acid 1 to about amino acid 21 of SEQ ID NO:7 and the sequence shown in SEQ ID NO:9; PSORT, Nakai, K., and Kanehisa, M., supra), which upon cleavage results in the production of a mature protein form. This mature protein form is approximately 252 amino acid residues in length (from about amino acid 22 to amino acid 273 of SEQ ID NO:7 and the sequence shown in SEQ ID NO:10).

A search of the murine FARS1 amino acid sequence against a database of profile HMM, e.g., Prosite (version 12.2), identified the following structural characteristics:

(i) two N-glycosylation sites (Prosite Accession Number PS00001) at about amino acids 100 to 103 and 162 to 165 of SEQ ID NO:2;

(ii) one cGMP-dependent protein kinase phosphorylation site (Prosite Accession Number PS00004) at about amino acids 232 to 235 of SEQ ID NO:2;

(iii) two protein kinase C phosphorylation sites (Prosite Accession Number PS00005) at about amino acids 227 to 229 and 246 to 248 of SEQ ID NO:2; two casein kinase II phosphorylation sites (Prosite Accession Number PS00006) at about amino acids 89 to 92 and 174 to 177 of SEQ ID NO:2; and

(iv) four N-myristoylation sites (Prosite Accession Number PS00008) at about amino acids 72 to 77, 96 to 101, 166 to 171, and 250 to 255 of SEQ ID NO:2.

The human and murine FARS1 sequences share a significant number of structural characteristics, as well as a nucleotide identity of 86% over the open reading frame and an amino acid sequence identity of 87%. Both the human and murine FARS1 amino acid sequences contain seven cysteine residues at about amino acids 11, 22, 35, 83, 109, 163, and 186 of SEQ ID NO:2 and SEQ ID NO:7. Additionally, both human and murine FARS1 are characterized by six dibasic cleavage sites at about amino acids R151-R152, K201-K202, K212-K213, K222-K223, K225-R226, and R232-K233 of SEQ ID NO:2 and SEQ ID NO:7. Processed peptides are involved in many aspects of body weight regulation and energy metabolism, e.g., as hormones (e.g., insulin), or as neuropeptides (e.g., NPY). Such peptides are synthesized and processed in a variety of tissues, including, e.g., pancreas, white adipose tissue, and hypothalamus.

The human and murine FARS1 genes and proteins represent members of a novel family of secreted proteins. The term “family” when referring to the protein and nucleic acid molecules of the invention means two or more-proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins (e.g., the polypeptide encoded by SEQ ID NO:6 or SEQ ID NO:8, or the amino acid sequence shown in SEQ ID NO:7 or SEQ ID NO:10). Members of a family can also have common functional characteristics.

A FARS1 protein can include a signal sequence. As used herein, a “signal peptide” or “signal sequence” refers to a peptide of about 12 to 60, preferably about 18 to 40, more preferably, 21 amino acid residues in length which occurs at the N-terminus of the protein and which contains a majority of hydrophobic amino acid residues. For example, a signal sequence contains at least about 12 to 60, preferably about 18 to 40, more preferably, 21 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a FARS1 protein contains a signal sequence of about 22 amino acids. The “signal sequence” is cleaved during processing of the mature protein. The mature FARS1 protein corresponds to amino acids 23 to 270 of SEQ ID NO:2 or amino acids 23 to 273 of SEQ ID NO:7.

A FARS1 polypeptide can include one or more of the following structural characteristics: (1) at least one, two, three, four, preferably five, more preferably six, most preferably seven cysteine residues; (2) at least one, two, three, preferably four, more preferably five, most preferably six dibasic proteolytic cleavage sites; at least one, preferably two, N-glycosylation sites; (3) at least one cGMP-dependent protein kinase phosphorylation site; (4) at least one, preferably two, protein kinase C phosphorylation sites; (5) at least one, preferably two, casein kinase II phosphorylation sites; or (6) at least one, two, preferably three, N-myristylation sites.

A FARS1 polypeptide can include a coiled coil structure. In human FARS1, the coiled coil structure is located from about amino acids 183 to 251 of SEQ ID NO:2. In murine FARS1, the coiled coil structure is located from about amino acids 183 to 254 of SEQ ID NO:7. Coiled coil-structures are supercoiled helical domains responsible for the oligomerization of proteins. There is a characteristic heptad repeat (h-x-x-h-x-x-x)n in the coiled coil structures, where h represents hydrophobic residues (Beck and Brodsky (1998) J. Struct. Biol. 122:17-29). Coiled coil structures are found in a wide variety of proteins, including cytoskeletal, nuclear, muscle, cell surface, extracellular, plasma, bacterial, and viral proteins.

A FARS1 polypeptide can include a bZIP transcription factor domain. As used herein, the term “bZIP transcription factor domain” or “bZIP domain” includes an amino acid sequence of about 20 to 50 amino acid residues in length, more preferably about 25 to 40 amino acid residues, and most preferably about 30 to 36 amino acids in length. Preferably, the domain includes the sequence at about amino acids 200 to 234 of SEQ ID NO:2. In another preferred embodiment, the bZIP transcription factor domain includes the sequenceat about amino acids 200 to 237 of SEQ ID NO:7. The bZIP transcription factor domain has been assigned PFAM Accession Number PF00170 (http://genome.wustl.edu/Pfam/html).

A FARS1 polypeptide can include a K-box region. As used herein, the term “K-box region” includes an amino acid sequence of about 50 to 120 amino acid residues in length, more preferably about 60 to 100 amino acid residues, and most preferably about 65 to 80 amino acids. Preferably, the domain includes the sequence at about amino acids 186 to 251 of SEQ ID NO:2. In another preferred embodiment, the K box region includes the sequence at about amino acids 186 to 251 of SEQ ID NO:7. The K-box region has been assigned the PFAM Accession Number PF01486 (http://genome.wustl.edu/Pfam/.html). The K-box reion is commonly associated with SRF-type transcription factors.

In a preferred embodiment, a FARS1 polypeptide or protein has a “bZIP transcription factor domain” or a region which includes at least about 20 to 50, more preferably about 25 to 40, and most preferably about 30 to 36 amino acid residues and has at least about 40%, 50%, 60%, 70%, 80%, 90%, 95%, 99%, or 100% homology with a “bZIP transcription factor domain,” e.g., the bZIP transcription factor domain of human FARS1 (e.g., residues 200 to 234 of SEQ ID NO:2) or the bZIP transcription factor domain of murine FARS1 (e.g., residues 200 to 237 of SEQ ID NO:7).

In another preferred embodiment, a FARS1 polypeptide or protein has a “K-box region” or a region which includes at least about 50 to 120, more preferably about 60 to 100, or 65 to 80 amino acid residues and has at least about 40%, 50%, 60%, 70% 80% 90% 95%, 99%, or 100% homology with a “K-box region,” e.g., the K-box region of human FARS1 (e.g., residues 186 to 251 of SEQ ID NO:2) or the K-box region of murine FARS1 (e.g., residues 186 to 251 of SEQ ID NO:7).

FARS1 expression was detected in adipose tissue. It was also found to be expressed at a high level in skeletal muscle, less in hypothalamus and heart, and at lower levels in liver and kidney. Thus, it is evident that FARS1 polypeptides can be involved in one or more biological processes which occur in these tissues. In particular, FARS1 can be involved in modulating growth, proliferation, survival, differentition, and activity of cells of these tissues. FARS1 can be critical for the proper function of many physiological systems, including CNS function, cardiovascular function, skeletal muscle function, energy metabolism (e.g., thermogenesis), and lipid metabolism. As a result, the FARS1 protein can have a critical function in one or more of the following physiological processes: (1) CNS function, (2) cardiovascular function, (3) skeletal muscle function, (4) enery metabolism (e.g., thermogenesis), or (5) lipid metabolism.

As the FARS1 mRNA is expressed in adipose tissue, skeletal muscle, hypothalamus, heart, liver, and kidney, the FARS1 nucleic acids and polypeptides of the present invention can be involved in disorders characterized by aberrant activity of these cells.

Examples of disorders associated with growth or metabolism of adipose tissue include, but are not limited to, rapid unexplained weight loss or weight gain, obesity, anorexia, bulimia, cachexia, diabetes, generalized or familial partial lipodystrophy (peripheral fat wasting), hypercholesterolemia and other cholesterol imbalances, hyperlipidemia, lipomatosis (e.g., multiple symmetric lipomatosis, pelvic lipomatosis, epidural lipomatosis), panniculitis (e.g., acute panniculitis, nodular panniculitis, adiposis dolorosa, and other diseases of adipose cell differentiation and proliferation, metabolic rate, and lipid metabolism.

Examples of disorders asociated with skeletal muscle include, but are not limited to, muscular dystrophy (including, e.g., Duchenne muscular dystrophy, Becker muscular dystrophy, limb-girdle muscular dystrophy, congenital muscular dystrophy, myotonic muscular dystrophy, facioscapulohumeral muscular dystrophy, and oculopharyngeal muscular dystrophy) and myopathy (including, e.g., central core disease, nemaline (rod) myopathy, and centronuclear (myotubular) myopathy).

Examples of disorders associated with brain tissue include, but are not limited to, neurodegenerative disorders, e.g., Alzheimer's disease, Pick's disease, Huntington's disease, Parkinson's and other Lewy diffuse body diseases, multiple sclerosis, amyothrophic lateral sclerosis, progressive supranuclear palsy, epilepsy, and Jakob-Creutzfieldt disease; psychiatric disorders, e.g., depression, schizophrenic disorders, Korsakoff's psychosis, mania, anxiety disorders, or phobic disorders; behavior, learning or memory disorders, e.g., amnesia or age-related memory loss; neurological disorders, e.g., migraine; hypothalamic dysfunction (e.g., misregulation of food intake and feeding behavior, thermoregulation, sleep-wake cycle, thirst, and autonomic nervous system function); and diseases of the hypothalamus (e.g. resulting from craniopharyngiomas, gliomas, meningiomas, granulomatous disease, germinomas, aneurysms of the internal carotid artery, hamartomas, ependymomas, and teratomas).

Examples of renal disorders include, but are not limited to, renal tubular disorders (including, e.g., autosomal dominant and recessive polycystic disease, tuberous sclerosis, Von Hippel-Lindau disease, and Fanconi syndrome), glomerulopathy (including, e.g., diabetic nephropathy, glomerulopathy (including, e.g., acute nephritic syndome, proliferative glomerulonephritis, polyarteritis nodosa, and ANCA-associated small-vessel vasculitis (including, e.g., Wegner's granulomatosis, Churg-Strauss variants of polyarteritis nodosa, and pauci-immune renal-limited glomerulonephritis).

As used herein, disorders involving the heart, or “cardiovascular disease” or a “cardiovascular disorder” includes a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects).

A cardiovasular disease or disorder also includes an endothelial cell disorder.

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Disorders which can be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, A1-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein can be used for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Examples of cellular proliferative and/or differentiative disorders include cancer, e.g., carcinoma, sarcoma, metastatic disorders, or hematopoietic neoplastic disorders, e.g., leukemias. A metastatic tumor can arise from a multitude of primary tumor types, including but not limited to those of prostate, colon, lung, breast and liver origin.

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Disorders which can be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, A1-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein can be used for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Thus, the FARS1 nucleic acids and polypeptides can act as novel diagnostic targets and therapeutic agents for controlling disorders in, e.g., cell growth, differentiation, and proliferation, neurotransmission, vascular tone, and the growth, differentiation, or proliferation or metabolism of the tissues in which it is expressed, e.g., adipose tissue, skeletal muscle, hypothalamus, heart, liver, and kidney.

Additionally, as the FARS1 polypeptides of the invention modulate FARS1-mediated activities, they can be useful for developing novel diagnostic and therapeutic agents for FARS1-mediated or associated disorders. For example, the FARS1 molecules can act as novel diagnostic targets and therapeutic agents, e.g., in controlling disorders of physiological function, such as lipid metabolism, energy metabolism, skeletal muscle function, cardiovascular function, CNS function, hepatic function, and renal function.

As used herein, a “FARS1 activity,” “biological activity of FARS1,” or “functional activity of FARS1,” refers to an activity exerted by a FARS1 protein, polypeptide or nucleic acid molecule on, e.g., a FARS1-responsive cell or on a FARS1 substrate, e.g., a protein substrate, as determined in vivo, in vitro, or in situ, according to standard techniques. In one embodiment, a FARS1 activity is a direct activity, such as an association (e.g., binding or interaction) with or an enzymatic activity exerted on a FARS1 target molecule. A “target molecule” or “binding partner” is a molecule with which a FARS1 protein binds or interacts in nature. A FARS1 activity includes an indirect activity, e.g., a cellular signaling activity mediated by interaction of the FARS1 protein with a FARS1 binding partner. Other activities include modulation of cellular proliferation, cellular differentiation, chemotaxis, cellular migration, cell death (e.g., apoptosis), or some combination of these.

The FARS1 protein, fragments thereof, and derivatives and other variants of the sequence in SEQ ID NO:2, SEQ ID NO:5, SEQ ID NO:7, OR SEQ ID NO:10 are collectively referred to as “polypeptides or proteins of the invention” or “FARS1 polypeptides or proteins.” Nucleic acid molecules encoding such polypeptides or proteins are collectively referred to as “nucleic acids of the invention” or “FARS1 nucleic acids.”FARS1 molecules refer to FARS1 nucleic acids, polypeptides, and antibodies.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules which are present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred example of stringent hybridization conditions is hybridization in 6×sodium chloride/sodium citrate (SSC) at about 45° C., followed by one or more washes in 0.2× SSC, 0.1% SDS at 50° C. Another example of stringent hybridization conditions is hybridization in 6× SSC at about 45° C., followed by one or more washes in 0.2× SSC, 0.1% SDS at 55° C. A further example of stringent hybridization conditions is hybridization in 6× SSC at about 45° C., followed by one or more washes in 0.2× SSC, 0.1% SDS at 60° C. Preferably, stringent hybridization conditions are hybridization in 6× SSC at about 45° C., followed by one or more washes in 0.2× SSC, 0.1% SDS at 65° C. Particularly preferred stringency conditions (and the conditions that should be used if the practitioner is uncertain about what conditions should be applied to determine if a molecule is within a hybridization limitation of the invention) are 0.5M sodium phosphate, 7% SDS at 65° C., followed by one or more washes at 0.2× SSC, 1% SDS at 65° C. Preferably, an isolated nucleic acid molecule of the invention that hybridizes under stringent conditions to the sequence SEQ ID NO:1, SEQ ID NO:3, SEQ ID NO:6, SEQ ID NO:8, or the sequence of the DNA insert of the plasmid deposited with ATCC Accession Number ______ corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a FARS1 protein, preferably a mammalian FARS1 protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of FARS1 protein having less than about 30%, 20%, 10% and more preferably 5% (by dry weight), of non-FARS1 protein (also referred to herein as a “contaminating protein”), or of chemical precursors or non-FARS1 chemicals. When the FARS1 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of FARS1 (e.g., the sequence of SEQ ID NO:2, SEQ ID NO:5, SEQ ID NO:7, SEQ ID NO:10, or polypeptide encoded by the nucleotide sequence of the DNA insert of the plasmid deposited with ATCC as Accession Number ______) without abolishing or, more preferably, without resulting in a substantial alteration of a biological activity, whereas an “essential” amino acid residue results in such a change. For example, amino acid residues that are conserved among the polypeptides of the present invention are predicted to be particularly unamenable to alteration.

The term “substantial alteration” is used herein to refer to an alteration of a FARS1 biological activity as detected by any of the methods described herein. For example, an alteration in an amino acid or nucleotide residue that results in a change in FARS1 activity that is at least 1%, 2%, 3%, 4%, 5%, 7%, preferably 10%, more preferably 15%, and most preferably 20% or more is defined herein as a substantial alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a FARS1 protein is preferably replaced with another amino acid residue from the same side chain family. Alternatively, in another embodiment, mutations can be introduced randomly along all or part of a FARS1 coding sequence, such as by saturation mutagenesis, and the resultant mutants can be screened for FARS1 biological activity to identify mutants that retain activity. Following mutagenesis of SEQ ID NO:1, SEQ ID NO:3, SEQ ID NO:6, SEQ ID NO:8, or the nucleotide sequence of the DNA insert of the plasmid deposited with ATCC as Accession Number ______, the encoded protein can be expressed recombinantly and the activity of the protein can be determined.

As used herein, a “biologically active portion” or “biologically active fragment” of a FARS1 protein includes a fragment of a FARS1 protein which participates in an interaction between a FARS1 molecule and a non-FARS1 molecule. Biologically active portions of a FARS1 protein include peptides comprising amino acid sequences sufficiently homologous to or derived from the amino acid sequence of the FARS1 protein, e.g., the amino acid sequence shown in SEQ ID NO:2, SEQ ID NO:5, SEQ ID NO:7, SEQ ID NO:10, or the polypeptide encoded by the the nucleotide sequence of the DNA insert of the plasmid deposited with ATCC as Accession Number ______ which include fewer amino acids than the full length FARS1 proteins, and exhibit at least one activity of a FARS1 protein. A biologically active portion of a FARS1 protein can be a polypeptide which is, for example, 10, 25, 50, 75, 100, 200 or more amino acids in length. Biologically active portions of a FARS1 protein can be used as targets for developing agents which modulate a FARS1 mediated activity.

Particular FARS1 polypeptides of the present invention have an amino acid sequence sufficiently or substantially identical to the amino acid sequence of SEQ ID NO:2, SEQ ID NO:5, SEQ ID NO:7, SEQ ID NO:10, or the polypeptide encoded by the the nucleotide sequence of the DNA insert of the plasmid deposited with ATCC as Accession Number ______. The term “sufficiently identical” or “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%. 91%, 92%, 93%, 94%, 95%, 96%, 97%, 98% or 99% identity are defined herein as sufficiently or substantially identical.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 20%, 30%, preferably at least 40%, more preferably at least 50%, even more preferably at least 60%, and even more preferably at least 70%, 80%, 90%, 100% of the length of the reference sequence (e.g., when aligning a second sequence to the FARS1 amino acid sequence of SEQ ID NO:2, SEQ ID NO:5, SEQ ID NO:7, SEQ ID NO:10, or the polypeptide encoded by the nucleotide sequence of the DNA insert of the plasmid deposited with ATCC as Accession Number ______. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller, (1989) CABIOS, 4:11-17, which has been incorporated into the ALIGN program (version 2.0), using a PAM 120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990) J. Mol. Biol. 215:403-10. BLAST nucleotide searches can be performed with the NBLAST program, score=100, wordlength=12 to obtain nucleotide sequences homologous to FARS1 nucleic acid molecules of the invention. BLAST protein searches can be performed with the XBLAST program, score=50, wordlength=3 to obtain amino acid sequences homologous to FARS1 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

“Misexpression or aberrant expression,” as used herein, refers to a non-wild type pattern of gene expression, at the RNA or protein level. It includes expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject,” as used herein, can refer to a mammal, e.g., a human, or to an experimental or animal or disease model. The subject can also be a non-human animal, e.g., a horse, cow, goat, or other domestic animal.

A “purified preparation of cells,” as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

Various aspects of the invention are described in further detail below.

Isolated Nucleic Acid Molecules

In one aspect, the invention provides, an isolated or purified, nucleic acid molecule that encodes a FARS1 polypeptide described herein, e.g., a full length FARS1 protein or a fragment thereof, e.g., a biologically active portion or fragment of FARS1 protein. Also included is a nucleic acid fragment suitable for use as a hybridization probe, which can be used, e.g., to identify a nucleic acid molecule encoding a polypeptide of the invention, FARS1 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:1, SEQ ID NO:6, or the nucleotide sequence of the DNA insert of the plasmid deposited with ATCC as Accession Number ______, or a portion of any of these nucleotide sequences. In one embodiment, the nucleic acid molecule includes sequences encoding the FARS1 protein (i.e., “the coding region” of SEQ ID NO:1, as shown in SEQ ID NO:3, or the coding region of SEQ ID NO:6, shown in SEQ ID NO:8), as well as 5′ or 3′ untranslated sequences. Alternatively, the nucleic acid molecule can include only the coding region of SEQ ID NO:1 (e.g., SEQ ID NO:3) or SEQ ID NO:6 (e.g., SEQ ID NO:8) and, e.g., no flanking sequences which normally accompany the subject sequence.

In another embodiment, an isolated nucleic acid molecule of the invention includes a nucleic acid molecule which is a complement of the nucleotide sequence shown in SEQ ID NO:1, SEQ ID NO:3, SEQ ID NO:6, SEQ ID NO:8, or the nucleotide sequence of the DNA insert of the plasmid deposited with ATCC as Accession Number ______, or a portion of any of these nucleotide sequences. In other embodiments, the nucleic acid molecule of the invention is sufficiently complementary to the nucleotide sequence shown in SEQ ID NO:1, SEQ ID NO:3, SEQ ID NO:6, SEQ ID NO:8, or the nucleotide sequence of the DNA insert of the plasmid deposited with ATCC as Accession Number ______ such that it can hybridize to the nucleotide sequence shown in in SEQ ID NO:1, SEQ ID NO:3, SEQ ID NO:6, SEQ ID NO:8, or the nucleotide sequence of the DNA insert of the plasmid deposited with ATCC as Accession Number ______, thereby forming a stable duplex.

In one embodiment, an isolated nucleic acid molecule of the present invention includes a nucleotide sequence which is at least about: 80%, 85%, 90%, 91%, 92%, 93%, 94%, 95%, 96%, 97%, 98%, 99%, or more homologous to the entire length of the nucleotide sequence shown in SEQ ID NO:1, SEQ ID NO:3, SEQ ID NO:6, SEQ ID NO:8, or the entire length of the nucleotide sequence of the DNA insert of the plasmid deposited with ATCC as Accession Number ______, or a portion, preferably of the same length, of any of these nucleotide sequences.

FARS1 Nucleic Acid Fragments

A nucleic acid molecule of the invention can include only a portion of the nucleic acid sequence of SEQ ID NO:1, SEQ ID NO:3, SEQ ID NO:6, SEQ ID NO:8, or the nucleotide sequence of the DNA insert of the plasmid deposited with ATCC as Accession Number ______. For example, such a nucleic acid molecule can include a fragment which can be used as a probe or primer or a fragment encoding a portion of a FARS1 protein, e.g., an immunogenic or biologically active portion of a FARS1 protein. A fragment can comprise those nucleotides of SEQ ID NO:1, SEQ ID NO:3, SEQ ID NO:6, SEQ ID NO:8, or the nucleotide sequence of the DNA insert of the plasmid deposited with ATCC as Accession Number ______ which encode a bZIP transcription factor domain or a K-box region of human FARS1. The nucleotide sequence determined from the cloning of the FARS1 gene allows for the generation of probes and primers designed for use in identifying and/or cloning other FARS1 family members, or fragments thereof, as well as FARS1 homologues, or fragments thereof, from other species.

In another embodiment, a nucleic acid includes a nucleotide sequence that includes part, or all, of the coding region and extends into either (or both) the 5′ or 3′ noncoding region. Other embodiments include-a fragment which includes a nucleotide sequence encoding an amino acid fragment described herein. Nucleic acid fragments can encode a specific domain or site described herein or fragments thereof, particularly fragments thereof which are at least 100 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a FARS1 nucleic acid fragment can include a sequence corresponding to a bZIP transcription factor domain or a K-box region.

FARS1 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO:1, SEQ ID NO:3, SEQ ID NO:6, SEQ ID NO:8, or the nucleotide sequence of the DNA insert of the plasmid deposited with ATCC as Accession Number ______, or of a naturally occurring allelic variant or mutant of SEQ ID NO:1, SEQ ID NO:3, SEQ ID NO:6, SEQ ID NO:8, or the nucleotide sequence of the DNA insert of the plasmid deposited with ATCC as Accession Number ______. In a preferred embodiment the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology.

“Looped” out sequences from deletions or insertions, or mismatches, are considered differences.

A probe or primer can be derived from the sense or anti-sense strand of a nucleic acid which encodes a bZIP transcription factor domain of FARS1 (from about amino acids 200 to 234 of SEQ ID NO:2 or from about amino acids 200 to 237 of SEQ ID NO:7) or a K-box region of FARS1 (from about amino acids 186 to 251 of SEQ ID NO:2 or from about amino acids 186 to 251).

In another embodiment a set of primers is provided, e.g., primers suitable for use in a PCR, which-can be used to amplify a selected region of a FARS1 sequence, e.g., a domain, region, site or other sequence described herein. The primers should be at least 5, 10, or 50 base pairs in length and less than 100, or less than 200, base pairs in length. The primers should be identical, or differ by one base from a sequence disclosed herein or from a naturally occurring variant. For example, primers suitable for amplifying all or a portion of any of the following regions are provided: a bZIP transcription factor domain of FARS1 (from about amino acids 200 to 234 of SEQ ID NO:2) or a K-box region of FARS1 (from about amino acids 186 to 251 of SEQ ID NO:2).

A nucleic acid fragment can encode an epitope bearing region of a polypeptide described herein.

A nucleic acid fragment encoding a “biologically active portion of a FARS1 polypeptide” or a “biologically active fragment of a FARS1 polypeptide” can be prepared by isolating a portion of the nucleotide sequence of SEQ ID NO:1, SEQ ID NO:3, SEQ ID NO:6, SEQ ID NO:8, or the nucleotide sequence of the DNA insert of the plasmid deposited with ATCC as Accession Number ______, which encodes a polypeptide having a FARS1 biological activity (e.g., the biological activities of the FARS1 proteins as described herein), expressing the encoded portion of the FARS1 protein (e.g., by recombinant expression in vitro) and assessing the activity of the encoded portion of the FARS1 protein. A nucleic acid fragment encoding a biologically active portion of a FARS1 polypeptide, may comprise a nucleotide sequence which is greater than 300 or more nucleotides in length.

In preferred embodiments, a nucleic acid includes a nucleotide sequence which is about 300, 400, 500, 600, 700, 800, 900, 1000, 1100, 1200, 1300 or more nucleotides in length and hybridizes under stringent hybridization conditions to a nucleic acid molecule of SEQ ID NO:1, or SEQ ID NO:3, or the nucleotide sequence of the DNA insert of the plasmid deposited with ATCC as Accession Number ______, or a complement thereof.

FARS1 Nucleic Acid Variants

The invention further encompasses nucleic acid molecules that differ from the nucleotide sequence shown in SEQ ID NO:1, SEQ ID NO:3, SEQ ID NO:6, SEQ ID NO:8, or the nucleotide sequence of the DNA insert of the plasmid deposited with ATCC as Accession Number ______. Such differences can be due to degeneracy of the genetic code (and result in a nucleic acid which encodes the same FARS1 proteins as those encoded by the nucleotide sequence disclosed herein). In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2, SEQ ID NO:5, SEQ ID NO:7, SEQ ID NO:10, or the polypeptide encoded by the nucleotide sequence of the DNA insert in the plasmid deposited with the ATCC as Accession Number ______. If alignment is needed for this comparison the sequences should be aligned for maximum homology. “Looped” out sequences from deletions or insertions, or mismatches, are considered differences.

Nucleic acids of the invention can be chosen for having codons, which are preferred, or nonpreferred, for a particular expression system. For example, the nucleic acid can be one in which at least one codon, at preferably at least 10%, or 20% of the codons has been altered such that the sequence is optimized for expression in bacterial, yeast, human, insect, or mammalian cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:1, SEQ ID NO:3, SEQ ID NO:6, SEQ ID NO:8, or the nucleotide sequence of the DNA insert in the plasmid deposited with the ATCC Accession Number ______, e.g., as follows: by at least one but less than 10, 20, 30, or 40 nucleotides; at least one but less than 1%, 5%, 10% or 20% of the subject nucleic acid. If necessary for this analysis, the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95%, or more identical to the nucleotide sequence shown in SEQ ID NO:1, SEQ ID NO:3, SEQ ID NO:6, SEQ ID NO:8, or the sequence in ATCC Accession Number ______, or a fragment of any of these sequences. Such nucleic acid molecules can readily be identified as being able to hybridize under stringent conditions to the nucleotide sequence shown SEQ ID NO:1, SEQ ID NO:3, SEQ ID NO:6, SEQ ID NO: 8, or the nucleotide sequence of the DNA insert in the plasmid deposited with the ATCC Accession Number ______, or a fragment of any of these sequences. Nucleic acid molecules corresponding to orthologs, homologs, and allelic variants of the FARS1 cDNAs of the invention can further be isolated by mapping to the same chromosome or locus as the FARS1 gene.

Preferred variants include those that are expressed in adipose tissue.

Allelic variants of FARS1, e.g., human FARS1, include both functional and non-functional proteins. Functional allelic variants are naturally occurring amino acid sequence variants of the FARS1 protein within a population that maintain the ability to bind substrates and hydrolyze them. Functional allelic variants will typically contain only conservative substitution of one or more amino acids of SEQ ID NO:2, SEQ ID NO:5, SEQ ID NO:7, SEQ ID NO:10, or the polypeptide encoded in the nucleotide sequence of the DNA insert in the plasmid deposited with the ATCC asAccession Number ______, or substitution, deletion or insertion of non-critical residues in non-critical regions of the protein. Non-functional allelic variants are naturally-occurring amino acid sequence variants of the FARS1, e.g., human FARS1, protein within a population that is not expressed in adipose tissue. Non-functional allelic variants will typically contain a non-conservative substitution, a deletion, or insertion, or premature truncation of the amino acid sequence of SEQ ID NO:2, SEQ II) NO:5, SEQ ID NO:7, SEQ ID NO:10, or the polypeptide encoded in the sequence in ATCC Accession Number ______, or a substitution, insertion, or deletion in critical residues or critical regions of the protein.

Moreover, nucleic acid molecules encoding other FARS1 family members and, thus, which have a nucleotide sequence which differs from the FARS1 sequences of SEQ ID NO:1, SEQ ID NO:3, SEQ ID NO:6, SEQ ID NO:8, or the nucleotide sequence of the DNA insert of the plasmid deposited with ATCC as Accession Number ______, are intended to be within the scope of the invention.

Antisense Nucleic Acid Molecules, Ribozymes and Modified FARS1 Nucleic Acid Molecules

In another aspect, the invention features, an isolated nucleic acid molecule which is antisense to FARS1. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire FARS1 coding strand, or to only a portion thereof (e.g., the coding region of FARS1 corresponding to SEQ ID NO:3 or SEQ ID NO:8). In another embodiment, the antisense nucleic acid molecule is antisense to a “noncoding region” of the coding strand of a nucleotide sequence encoding FARS1 (e.g., the 5′ and 3′ untranslated regions).

An antisense nucleic acid can be designed such that it is complementary to the entire coding region of FARS1 mRNA, but more preferably is an oligonucleotide which is antisense to only a portion of the coding or noncoding region of FARS1 mRNA. For example, the antisense oligonucleotide can be complementary to the region surrounding the translation start site of FARS1 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a FARS1 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or po III promoter are preferred.

In yet another embodiment, the antisense nucleic acid molecule of the invention is an I-anomeric nucleic acid molecule. An I-anomeric nucleic acid molecule forms specific double-stranded hybrids with complementary RNA in which, contrary to the usual θ-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a FARS1-encoding nucleic acid can include one or more sequences complementary to the nucleotide sequence of a FARS1 cDNA disclosed herein (i.e., SEQ ID NO:1, SEQ ID NO:3, SEQ ID NO:6, SEQ ID NO:8, or the nucleotide sequence of the DNA insert of the plasmid deposited with ATCC as Accession Number ______), and a sequence having known catalytic sequence responsible for mRNA cleavage (see U.S. Pat. No. 5,093,246 or Haselhoff and Gerlach, (1988) Nature 334:585-591). For example, a derivative of a Tetrahymena L-19 UVS RNA can be constructed in which the nucleotide sequence of the active site is complementary to the nucleotide sequence to be cleaved in a FARS1-encoding mRNA. See, e.g., Cech, et al., U.S. Pat. No. 4,987,071; and Cech, et al., U.S. Pat. No. 5,116,742. Alternatively, FARS1 mRNA can be used to select a catalytic RNA having a specific ribonuclease activity from a pool of RNA molecules. See, e.g., Bartel, D. and Szostak, J. W. (1993) Science 261:1411-1418.

FARS1 gene expression can be inhibited by targeting nucleotide sequences complementary to the regulatory region of the FARS1 (e.g., the FARS1 promoter and/or enhancers) to form triple helical structures that prevent transcription of the FARS1 gene in target cells. See, generally, Helene, C. (1991) Anticancer Drug Des. 6:569-84; Helene, C., et al. (1992) Ann. N.Y. Acad. Sci. 660:27-36; and Maher, L. J. (1992) Bioassays 14:807-15. The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the-necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A FARS1 nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B., et al. (1996) supra; Perry-OKeefe, et al. Proc. Natl. Acad. Sci. USA 93:14670-675.

PNAs of FARS1 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of FARS1 nucleic acid molecules can also be used in the analysis of single base pair mutations in a gene, (e.g., by PNA-directed PCR clamping); as ‘artificial restriction enzymes’ when used in combination with other enzymes, (e.g., SI nucleases (Hyrup B., supra)); or as probes or primers for DNA sequencing or hybridization (Hyrup B. et al., supra; Perry-O'Keefe, et al., supra).

In other embodiments, the oligonucleotide may include other appended groups such as peptides (e.g., for targeting host cell receptors in vivo), or agents facilitating transport across the cell membrane (see, e.g., Letsinger, et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a FARS1 nucleic acid of the invention, two complementary regions one having a fluorophore and one a quencher such that the molecular beacon is useful for quantitating the presence of the FARS1 nucleic acid of the invention in a sample. Molecular beacon nucleic acids are described, for example, in Lizardi, et al., U.S. Pat. No. 5,854,033; Nazarenko, et al., U.S. Pat. No. 5,866,336, and Livak, et al., U.S. Pat. No. 5,876,930.

Isolated FARS1 Polypeptides

In another aspect, the invention features an isolated FARS1 protein, or fragment, e.g., a biologically active portion, for use as immunogens or antigens to raise or test (or more generally to bind) anti-FARS1 antibodies. FARS1 protein can be isolated from cells or tissue sources using standard protein purification techniques. FARS1 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a FARS1 polypeptide has one or more of the following characteristics:

(i) the ability to modulate one or more of cell growth, differentiation, adherence, motility, or intercellular interactions;

(ii) a molecular weight, e.g., a deduced molecular weight, preferably ignoring any contribution of post translational modifications, amino acid composition or other physical characteristic of SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______;

(iii) an overall sequence similarity of at least 80%, more preferably at least 90, or 95%, with a polypeptide of SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______;

(iv) a bZIP transcription factor domain which is preferably about 70%, 80%, 90% or 95% homologous with amino acid residues about 200 to 234 of SEQ ID NO:2; or

(v) a K-box region which is preferably about 70%, 80%, 90% or 95% homologous with amino acid residues about 186 to 251 of SEQ ID NO:2.

In a preferred embodiment, the FARS1 protein, or fragment thereof, differs from the corresponding sequence in SEQ ID:2 or SEQ ID NO:6. In one embodiment it differs by at least one but by less than 15, 10 or 5 amino acid residues. In another it differs from the corresponding sequence in SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______ by at least one residue but less than 20%, 15%, 10% or 5% of the residues in it differ from the corresponding sequence in SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______. (If this comparison requires alignment, the sequences should be aligned for maximum homology. “Looped” out sequences from deletions or insertions, or mismatches, are considered differences.) The differences are, preferably, differences or changes at a non essential residue or a conservative substitution. In a preferred embodiment, the differences are not in the bZIP transcription factor domain (at about amino acids 200 to 237 of SEQ ID NO:2 or at about amino acids 200 to 234 of SEQ ID NO:7). In another preferred embodiment, the differences are not in the K-box region (at about amino acids 186 to 251 of SEQ ID NO:2 or at about amino acids 186 to 251 of SEQ ID NO:7).

Other embodiments include a protein that contain one or more changes in amino acid sequence, e.g., a change in an amino acid residue which is not essential for activity. Such FARS1 proteins differ in amino acid sequence from SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______ yet retain biological activity.

In one embodiment, the protein includes an amino acid sequence at least about 80%, 85%, 90%, 95%, 98% or more homologous to SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______.

In one embodiment, a FARS1 protein- or fragment is provided which varies from the sequence of SEQ ID NO.2 in regions defined by amino acids about 1 to 185 and 252 to 270 by at least one but by less than 15, 10 or 5 amino acid residues in the protein or fragment but which does not differ from SEQ ID NO.2 in regions defined by amino acids about 186 to 251. In another embodiment, a FARS1 protein or fragment is provided which varies from the sequence of SEQ ID NO.7 in regions defined by amino acids about 1 to 185 and 252 to 273 by at least one but by less than 15, 10 or 5 amino acid residues in the protein or fragment but which does not differ from SEQ ID NO.2 in regions defined by amino acids about 186 to 251. If this comparison requires alignment the sequences should be aligned for maximum homology. “Looped” out sequences from deletions or insertions, or mismatches, are considered differences. In some embodiments the difference is at a non essential residue or is a conservative substitution, while in others the difference is at an essential residue or is a non conservative substitution.

In one embodiment, a biologically active portion of a FARS1 protein includes a bZIP transcription factor domain. In another embodiment, a biologically active portion of a FARS1 protein includes a K-box region. Moreover, other biologically active portions, in which other regions of the protein are deleted, can be prepared by recombinant techniques and evaluated for one or more of the functional activities of a native FARS1 protein.

In a preferred embodiment, the FARS1 protein has an amino acid sequence shown in SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______. In other embodiments, the FARS1 protein is substantially identical to SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______. In yet another embodiment, the FARS1 protein is substantially identical to SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______ and retains the functional activity of the protein of SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______, as described in detail in the subsections above.

FARS1 Chimeric or Fusion Proteins

In another aspect, the invention provides FARS1 chimeric or fusion proteins. As used herein, a FARS1 “chimeric protein” or “fusion protein” includes a FARS1 polypeptide linked to a non-FARS1 polypeptide. A “non-FARS1 polypeptide” refers to a polypeptide having an amino acid sequence corresponding to a protein which is not substantially homologous to the FARS1 protein, e.g., a protein which is different from the FARS1 protein and which is derived from the same or a different organism. The FARS1 polypeptide of the fusion protein can correspond to all or a portion e.g., a fragment described herein of a FARS1 amino acid sequence. In a preferred embodiment, a FARS1 fusion protein includes at least one (or two) biologically active portion of a FARS1 protein. The non-FARS1 polypeptide can be fused to the N-terminus or C-terminus of the FARS1 polypeptide.

The fusion protein can include a moiety which has a high affinity for a ligand. For example, the fusion protein can be a GST-FARS1 fusion protein in which the FARS1 sequences are fused to the C-terminus of the GST (glutathione S-transferase) sequences. Such fusion proteins can facilitate the purification of recombinant FARS1. Alternatively, the fusion protein can be a FARS1 protein containing a heterologous signal sequence at its N-terminus. In certain host cells (e.g., mammalian host cells), expression and/or secretion of FARS1 can be increased through use of a heterologous signal sequence.

Fusion proteins can include all or a part of a serum protein, e.g., an IgG constant region, or human serum albumin.

The FARS1 fusion proteins of the invention can be incorporated into pharmaceutical compositions and administered to a subject in vivo. The FARS1 fusion proteins can be used to affect the bioavailability of a FARS1 substrate. FARS1 fusion proteins may be useful therapeutically for the treatment of disorders caused by, for example, (i) aberrant modification or mutation of a gene encoding a FARS1 protein; (ii) mis-regulation of the FARS1 gene; and (iii) aberrant post-translational modification of a FARS1 protein.

Moreover, the FARS1-fusion proteins of the invention can be used as immunogens to produce anti-FARS1 antibodies in a subject, to purify FARS1 ligands and in screening assays to identify molecules which inhibit the interaction of FARS1 with a FARS1 substrate.

Expression vectors are commercially available that already encode a fusion moiety (e.g., a GST polypeptide). A FARS1-encoding nucleic acid can be cloned into such an expression vector such that the fusion moiety is linked in-frame to the FARS1 protein.

Variants of FARS1 Proteins

In another aspect, the invention also features a variant of a FARS1 polypeptide, e.g., which functions as an agonist (mimetics) or as an antagonist. Variants of the FARS1 proteins can be generated by mutagenesis, e.g., discrete point mutation, the insertion or deletion of sequences or the truncation of a FARS1 protein. An agonist of the FARS1 proteins can retain substantially the same, or a subset, of the biological activities of the naturally occurring form of a FARS1 protein. An antagonist of a FARS1 protein can inhibit one or more of the activities of the naturally occurring form of the FARS1 protein by, for example, competitively modulating a FARS1-mediated activity of a FARS1 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the FARS1 protein.

Variants of a FARS1 protein can be identified by screening combinatorial libraries of mutants, e.g., truncation mutants, of a FARS1 protein for agonist or antagonist activity.

Libraries of fragments e.g., N terminal, C terminal, or internal fragments, of a FARS1 protein coding sequence can be used to generate a variegated population of fragments for screening and subsequent selection of variants of a FARS1 protein.

Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Recursive ensemble mutagenesis (REM), a new technique which enhances the frequency of functional mutants in the libraries, can be used in combination with the screening assays to identify FARS1 variants (Arkin and Yourvan (1992) Proc. Natl. Acad. Sci. USA 89:7811-7815; Delgrave et al. (1993) Protein Engineering 6(3):327-331).

Cell based assays can be exploited to analyze a variegated FARS1 library. For example, a library of expression vectors can be transfected into a cell line, e.g., a cell line, which ordinarily responds to FARS1 in a substrate-dependent manner. The transfected cells are then contacted with FARS1 and the effect of the expression of the mutant on signaling by the FARS1 substrate can be detected. Plasmid DNA can then be recovered from the cells which score for inhibition, or alternatively, potentiation of signaling by the FARS1 substrate, and the individual clones further characterized.

In another aspect, the invention features a method of making a FARS1 polypeptide, e.g., a peptide having a non-wild type activity, e.g., an antagonist, agonist, or super agonist of a naturally occurring FARS1 polypeptide, e.g., a naturally occurring FARS1 polypeptide. The method includes: altering the sequence of a FARS1 polypeptide, e.g., altering the sequence by substitution or deletion of one or more residues of a non-conserved region, a domain or residue disclosed herein, and testing the altered polypeptide for the desired activity.

In another aspect, the invention features a method of making a fragment or analog of a FARS1 polypeptide possessing a biological activity of a naturally occurring FARS1 polypeptide. The method includes altering the sequence, e.g., by substitution or deletion of one or more residues, of a FARS1 polypeptide, e.g., altering the sequence of a non-conserved region, or a domain or residue described herein, and testing the altered polypeptide for the desired activity.

Anti-FARS1 Antibodies

In another aspect, the invention provides an anti-FARS1 antibody. The term “antibody” as used herein refers to an immunoglobulin molecule or immunologically active portion thereof, i.e., an antigen-binding portion. Examples of immunologically active portions of immunoglobulin molecules include scFV and dcFV fragments, Fab and F(ab′)₂ fragments which can be generated by treating the antibody with an enzyme such as papain or pepsin, respectively.

The antibody can be a polyclonal, monoclonal, recombinant, e.g., a chimeric or humanized, fully human, non-human, e.g., murine, or single chain antibody. In a preferred embodiment, the antibody has effector function and can fix complement. The antibody can be coupled to a heterologous molecule, e.g., a toxin or an imaging agent.

A full-length FARS1 protein or, antigenic peptide fragment of FARS1 can be used as an immunogen or can be used to identify anti-FARS1 antibodies made with other immunogens, e.g., cells, membrane preparations, and the like. The antigenic peptide of FARS1 should include at least 8 amino acid residues of the amino acid sequence shown in SEQ ID NO:2, SEQ ID NO:5, SEQ ID NO:7, SEQ ID NO:10, or the polypeptide encoded by the nucleotide sequence of the DNA insert of the plasmid deposted with the ATCC as Accession Number ______ and encompasses an epitope of FARS1. Preferably, the antigenic peptide includes at least 10 amino acid residues, more preferably at least 15 amino acid residues, even more preferably at least 20 amino acid residues, and most preferably at least 30 amino acid residues.

Fragments of FARS1 can be used to make, e.g., used as immunogens or used to characterize the specificity of an antibody, antibodies against hydrophilic regions of the FARS1 protein. Similarly, a fragment of FARS1 which includes residues about 1 to 21 of SEQ ID NO:2 (SEQ ID NO:4) or residues about 1 to 21 of SEQ ID NO:7 (SEQ ID NO:9) can be used to make an antibody against a hydrophobic region of the FARS1 protein; a fragment of FARS1 which includes residues about 200 to 234 of SEQ ID NO:2 or residues about 200 to 237 of SEQ ID NO:7 can be used to make an antibody against the bZIP transcription factor domain of the FARS1 protein; and a fragment of FARS1 which includes residues about 186 to 251 of SEQ ID NO:2 or residues about 186 to 251 of SEQ ID NO:7 can be used to make an antibody against the K-box region of the FARS1 protein.

Antibodies reactive with, or specific for, any of these regions, or other regions or domains described herein are provided.

Preferred epitopes encompassed by the antigenic peptide are regions of FARS1 are located on the surface of the protein, e.g., hydrophilic regions, as well as regions with high antigenicity. For example, an Emini surface probability analysis of the human FARS1 protein sequence can be used to indicate the regions that have a particularly high probability of being localized to the surface of the FARS1 protein and are thus likely to constitute surface residues useful for targeting antibody production.

In a preferred embodiment the antibody binds an epitope on any domain or region on FARS1 proteins described herein.

Additionally, chimeric, humanized, and completely human antibodies are also within the scope of the invention. Chimeric, humanized, but most preferably, completely human antibodies are desirable for applications which include repeated administration, e.g., therapeutic treatment (and some diagnostic applications) of human patients.

Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

The anti-FARS1 antibody can be a single chain antibody. A single-chain antibody (scFV) may be engineered (see, for example, Colcher, D., et al. Ann NY Acad Sci Jun. 30, 1999;880:263-80; and Reiter, Y. Clin Cancer Res 1996 February;2(2):245-52). The single chain antibody can be dimerized or multimerized to generate multivalent antibodies having specificities for different epitopes of the same target FARS1 protein.

In a preferred embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

An antibody (or fragment thereof) can be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Radioactive ions include, but are not limited to iodine, yttrium and praseodymium.

The conjugates of the invention can be used for modifying a given biological response, the therapeutic moiety is not to be construed as limited to classical chemical therapeutic agents. For example, the therapeutic moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“1L-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

An anti-FARS1 antibody (e.g., monoclonal antibody) can be used to isolate FARS1 by standard techniques, such as affinity chromatography or immunoprecipitation. Moreover, an anti-FARS1 antibody can be used to detect FARS1 protein (e.g., in a cellular lysate or cell supernatant) in order to evaluate the abundance and pattern of expression of the protein. Anti-FARS1 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵¹¹³I, ³⁵S or ³H.

In preferred embodiments, an antibody can be made by immunizing with a purified FARS1 antigen, or a fragment thereof, e.g., a fragment described herein, a membrane associated antigen, tissues, e.g., crude tissue preparations, whole cells, preferably living cells, lysed cells, or cell fractions, e.g., membrane fractions.

Antibodies which bind only a native FARS1 protein, only denatured or otherwise non-native FARS1 protein, or which bind both, are within the invention. Antibodies with linear or conformational epitopes are within the invention. Conformational epitopes sometimes can be identified by identifying antibodies which bind to native but not denatured FARS1 protein.

Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a FARS1 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., FARS1 proteins, mutant forms of FARS1 proteins, fusion proteins, and the like).

The recombinant expression vectors of the invention can be designed for expression of FARS1 proteins in prokaryotic or eukaryotic cells. For example, polypeptides of the invention can be expressed in bacterial, insect cells (e.g., using baculovirus expression vectors), yeast cells or mammalian cells. Suitable host cells are discussed further in Goeddel (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in FARS1 activity assays, (e.g., direct assays or competitive assays described in detail below), or to generate antibodies specific for FARS1 proteins. In a preferred embodiment, a-fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in prokaryotes, it is desirable to express the protein in a host bacteria, e.g., E. coli, with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized by the host bacteria, e.g., E. coli (Wada, et al., (1992) Nucleic Acids Res. 20:2111-2118). Such alteration of nucleic acid sequences of the invention can be carried out by standard DNA synthesis techniques.

The FARS1 expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the a-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes, see Weintraub, H. et al., (1986) Reviews—Trends in Genetics 1:1.

Another aspect the invention provides a host cell which includes a nucleic acid molecule described herein, e.g., a FARS1 nucleic acid molecule within a recombinant expression vector or a FARS1 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a FARS1 protein can be expressed in bacterial cells, such as E. coli, insect cells, yeast or mammalian cells (such as CHO or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a FARS1 protein. Accordingly, the invention further provides methods for producing a FARS1 protein using the host cells of the invention. In one embodiment, the method includes culturing the host cell of the invention (into which a recombinant expression vector encoding a FARS1 protein has been introduced) in a suitable medium such that a FARS1 protein is produced. In another embodiment, the method further includes isolating a FARS1 protein from the medium or the host cell.

In another aspect, the invention features, a cell or purified preparation of cells which include a FARS1 transgene, or which otherwise misexpress FARS1. The cell preparation can consist of human or nonhuman cells, e.g., rodent cells, e.g., mouse or rat cells, rabbit cells, or pig cells. In preferred embodiments, the cell or cells include a FARS1 transgene, e.g., a heterologous form of a FARS1, e.g., a gene derived from humans (in the case of a non-human cell). The FARS1 transgene can be misexpressed, e.g., overexpressed or underexpressed. In other preferred embodiments, the cell, or cells, includes a gene which misexpresses an endogenous FARS1, e.g., a gene the expression of which is disrupted, e.g., a knockout. Such cells can serve as a model for studying disorders which are related to mutated or mis-expressed FARS1 alleles or for use in drug screening.

In another aspect, the invention features a human cell, e.g., a hematopoietic stem cell, transformed with nucleic acid which encodes a subject FARS1 polypeptide.

Also provided are cells, preferably human cells, e.g., human hematopoietic or fibroblast cells, in which an endogenous FARS1 is under the control of a regulatory sequence that does not normally control the expression of the endogenous FARS1 gene. The expression characteristics of an endogenous gene within a cell, e.g., a cell line or microorganism, can be modified by inserting a heterologous DNA regulatory element into the genome of the cell such that the inserted regulatory element is operably linked to the endogenous FARS1 gene. For example, an endogenous FARS1 gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

Transgenic Animals

The invention provides non-human transgenic animals. Such animals are useful for studying the function and/or activity of a FARS1 protein and for identifying and/or evaluating modulators of FARS1 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or. a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous FARS1 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a FARS1 protein to particular cells. A transgenic founder animal can be identified based upon the presence of a FARS1 transgene in its genome and/or expression of FARS1 mRNA in tissues or cells of the animals. A transgenic founder animal can then be used to breed additional animals carrying the transgene. Moreover, transgenic animals carrying a transgene encoding a FARS1 protein can further be bred to other transgenic animals carrying other transgenes.

FARS1 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

Uses

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a FARS1 protein (e.g., via a recombinant expression vector in a host cell in gene therapy applications), to detect a FARS1 mRNA (e.g., in a biological sample) or a genetic alteration in a FARS1 gene, and to modulate FARS1 activity, as described further below. The FARS1 proteins can be used to treat disorders characterized by insufficient or excessive production of a FARS1 substrate or production of FARS1 inhibitors. In addition, the FARS1 proteins can be used to screen for naturally occurring FARS1 substrates, to screen for drugs or compounds which modulate FARS1 activity, as well as to treat disorders characterized by insufficient or excessive production of FARS1 protein or production of FARS1 protein forms which have decreased, aberrant or unwanted activity compared to FARS1 wild type protein (e.g., aberrant or deficient lipid metabolism, energy metabolism, skeletal muscle function, cardiovascular function, CNS function, hepatic function, and renal function). Moreover, the anti-FARS1 antibodies of the invention can be used to detect and isolate FARS1 proteins, regulate the bioavailability of FARS1 proteins, and modulate FARS1 activity.

A method of evaluating a compound for the ability to interact with, e.g., bind, a subject FARS1 polypeptide is provided. The method includes: contacting the compound with the subject FARS1 polypeptide; and evaluating ability of the compound to interact with, e.g., to bind or form a complex with the subject FARS1 polypeptide. This method can be performed in vitro, e.g., in a cell free system, or in vivo, e.g., in a two-hybrid interaction trap assay. This method can be used to identify naturally occurring molecules which interact with subject FARS1 polypeptide. It can also be used to find natural or synthetic inhibitors of subject FARS1 polypeptide. Screening methods are discussed in more detail below.

Screening Assays:

The invention provides methods (also referred to herein as “screening assays”) for identifying modulators, i.e., candidate or test compounds or agents (e.g., proteins, peptides, peptidomimetics, peptoids, small molecules or other drugs) which bind to FARS1 proteins, have a stimulatory or inhibitory effect on, for example, FARS1 expression or FARS1 activity, or have a stimulatory or inhibitory effect on, for example, the expression or activity of a FARS1 substrate. Compounds thus identified can be used to modulate the activity of target gene products (e.g., FARS1 genes) in a therapeutic protocol, to elaborate the biological function of the target gene product, or to identify compounds that disrupt normal target gene interactions.

In one embodiment, the invention provides assays for screening candidate or test compounds which are substrates of a FARS1 protein or polypeptide or a biologically active portion thereof. In another embodiment, the invention provides assays for screening candidate or test compounds which bind to or modulate the activity of a FARS1 protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37: 2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria and spores (U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. USA 87:6378-6382; Felici (1991) J. Mol. Biol. 222:301-310; and (U.S. Pat. No. 5,223,409).

In one embodiment, an assay is a cell-based assay in which a cell which expresses a FARS1 protein, or biologically active portion thereof, is contacted with a test compound, and the ability of the test compound to modulate FARS1 activity is determined. Determining the ability of the test compound to modulate FARS1 activity can be accomplished by comparing, for example, the interaction of FARS1 with its target molecule in the presence of the test compound with the interaction in the absence of the test compound. The cell, for example, can be of mammalian origin, e.g., human.

The ability of the test compound to modulate FARS1 binding to a compound, e.g., a FARS1 substrate, or to bind to FARS1 can also be evaluated. This can be accomplished, for example, by coupling the compound, e.g., the substrate, with a radioisotope or enzymatic label such that binding of the compound; e.g., the substrate, to FARS1 can be determined by detecting the labeled compound, e.g., substrate, in a complex. Alternatively, FARS1 could be coupled with a radioisotope or enzymatic label to monitor the ability of a test compound to modulate FARS1 binding to a FARS1 substrate in a complex. For example, compounds (e.g., FARS1 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a FARS1 substrate) to interact with FARS1 with or without the labeling of any of the interactants can be evaluated. For example, a microphysiometer can be used to detect the interaction of a compound with FARS1 without the labeling of either the compound or the FARS1. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and FARS1.

In yet another embodiment, a cell-free assay is provided in which a FARS1 protein or biologically active portion thereof is contacted with a test compound and the ability of the test compound to bind to the FARS1 protein or biologically active portion thereof is evaluated. Preferred biologically active portions of the FARS1 proteins to be used in assays of the present invention include fragments which participate in interactions with non-FARS1 molecules, e.g., fragments with high surface probability scores.

Soluble and/or membrane-bound forms of isolated proteins (e.g., FARS1 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the FARS1 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly; with detectable labels discussed herein.

It may be desirable to immobilize either FARS1, an anti-FARS1 antibody, or its target molecule to facilitate separation of complexed from uncomplexed forms of one or both of the proteins, as well as to accommodate automation of the assay. Binding of a test compound to a FARS1 protein, or interaction of a FARS1 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/FARS1 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or FARS1 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of FARS1 binding or activity determined using standard techniques.

Other techniques for immobilizing either a FARS1 protein or a target molecule on matrices include using conjugation of biotin and streptavidin. Biotinylated FARS1 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemicals).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the-antibody, in turn, can be directly labeled or indirectly labeled with, e.g., a labeled anti-Ig antibody).

In one embodiment, this assay is performed utilizing antibodies reactive with FARS1 protein or target molecules but which do not interfere with binding of the FARS1 protein to its target molecule. Such antibodies can be derivatized to the wells of the plate, and unbound target or FARS1 protein trapped in the wells by antibody conjugation. Methods for detecting such complexes, in addition to those described above for the GST-immobilized complexes, include immunodetection of complexes using antibodies reactive with the FARS1 protein or target molecule, as well as enzyme-linked assays which rely on detecting an enzymatic activity associated with the FARS1 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P. (1993) Trends Biochem. Sci. 18:284-7); chromatography (gel filtration chromatography, ion-exchange chromatography); electrophoresis (see, e.g., Ausubel, F. et al., eds. Current Protocols in Molecular Biology 1999, J. Wiley. New York.); and immunoprecipitation (see, e.g., Ausubel, F. et al., supra). Such resins and chromatographic techniques are known to one skilled in the art (see, e.g., Heegaard, N. H. (1998) J. Mol. Recognit. 11:141-8; Hage, D. S. and Tweed, S. A. (1997) J. Chromatogr. B. Biomed. Sci. Appl. 699:499-525). Further, fluorescence energy transfer may also be conveniently utilized, as described herein, to detect binding without further purification of the complex from solution.

In a preferred embodiment, the assay includes contacting the FARS1 protein or biologically active portion thereof with a known compound which binds FARS1 to form an assay mixture, contacting the assay mixture with a test compound, and determining the ability of the test compound to interact with a FARS1 protein, wherein determining the ability of the test compound to interact with a FARS1 protein includes determining the ability of the test compound to preferentially bind to FARS1 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the FARS1 genes herein identified. In an alternative embodiment, the invention provides methods for determining the ability of the test compound to modulate the activity of a FARS1 protein through modulation of the activity of a downstream effector of a FARS1 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner.

Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the FARS1 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with FARS1 (“FARS1-binding proteins” or “FARS1-bps”) and are involved in FARS1 activity. Such FARS1-bps can be activators or inhibitors of signals by the FARS1 proteins or FARS1 targets as, for example, downstream elements of a FARS1-mediated signaling pathway.

The two-hybrid system is based on the modular nature of most transcription factors, which consist of separable DNA-binding and activation domains. Briefly, the assay utilizes two different DNA constructs. In one construct, the gene that codes for a FARS1 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively FARS1 protein can be the fused to the activator domain.) If the “bait” and the “prey” proteins are able to interact, in vivo, forming a FARS1-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the FARS1 protein.

In another embodiment, modulators of FARS1 expression are identified. For example, a cell or cell free mixture is contacted with a candidate compound and the expression of FARS1 mRNA or protein evaluated relative to the level of expression of FARS1 mRNA or protein in the absence of the candidate compound. When expression of FARS1 mRNA or protein is greater in the presence of the candidate compound than in its absence, the candidate compound is identified as a stimulator of FARS1 mRNA or protein expression. Alternatively, when expression of FARS1 mRNA or protein is less (statistically significantly less) in the presence of the candidate compound than in its absence, the candidate compound is identified as an inhibitor of FARS1 mRNA or protein expression. The level of FARS1 mRNA or protein expression can be determined by methods described herein for detecting FARS1 mRNA or protein.

In another aspect, the invention pertains to a combination of two or more of the assays described herein. For example, a modulating agent can be identified using a cell-based or a cell free assay, and the ability of the agent to modulate the activity of a FARS1 protein can be confirmed in vivo, e.g., in an animal, such as an animal model for lipid metabolism, energy metabolism, skeletal muscle function, cardiovascular function, CNS function, hepatic function, renal function, and cell growth and differentiative disorders.

This invention further pertains to novel agents identified by the above-described screening assays. Accordingly, it is within the scope of this invention to further use an agent identified as described herein (e.g., a FARS1 modulating agent, an antisense FARS1 nucleic acid molecule, a FARS1-specific antibody, or a FARS1-binding partner) in an appropriate animal model to determine the efficacy, toxicity, side effects, or mechanism of action, of treatment with such an agent. Furthermore, novel agents identified by the above-described screening assays can be used for treatments as described herein.

Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate FARS1 with a disease; (ii) identify an individual from a minute biological sample (tissue typing); and (iii) aid in forensic identification of a biological sample. These applications are described in the subsections below.

Chromosome Mapping

The FARS1 nucleotide sequences or portions thereof can be used to map the location of the FARS1 genes on a chromosome. This process is called chromosome mapping. Chromosome mapping is useful in correlating the FARS1 sequences with genes associated with disease.

Briefly, FARS1 genes can be mapped to chromosomes by preparing PCR primers (preferably 15-25 bp in length) from the FARS1 nucleotide sequences. These primers can then be used for PCR screening of somatic cell hybrids containing individual human chromosomes. Only those hybrids containing the human gene corresponding to the FARS1 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (DEustachio P. et al. (1983) Science 220:919-924).

Other mapping strategies, e.g., in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries can be used to map FARS1 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., (1988) Human Chromosomes: A Manual of Basic Techniques, Pergamon Press, New York).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the FARS1 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

FARS1 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the FARS1 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 can provide positive individual identification with a panel of perhaps 10 to 1,000 primers which each yield a noncoding amplified sequence of 100 bases. If predicted coding sequences, such as those in SEQ ID NO:3 are used, a more appropriate number of primers for positive individual identification would be 500-2,000.

If a panel of reagents from FARS1 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial FARS1 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 (e.g., fragments derived from the noncoding regions of SEQ ID NO:1 having a length of at least 20 bases, preferably at least 30 bases) are particularly appropriate for this use.

The FARS1 nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such FARS1 probes can be used to identify tissue by species and/or by organ type.

In a similar fashion, these reagents, e.g., FARS1 primers or probes can be used to screen tissue culture for contamination (i.e. screen for the presence of a mixture of different types of cells in a culture).

Predictive Medicine

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes FARS1.

Such disorders include, e.g., disorders associated with the misexpression of FARS1 gene; CNS disorders, cardiovascular disorders, metabolic disorders, and cell growth and differentiative disorders.

The method includes one or more of the following:

detecting, in a tissue of the subject, the presence or absence of a mutation which affects the expression of the FARS1 gene, or detecting the presence or absence of a mutation in a region which controls the expression of the gene, e.g., a mutation in the 5′ control region;

detecting, in a tissue of the subject, the presence or absence of a mutation which alters the structure of the FARS1 gene;

detecting, in a tissue of the subject, the misexpression of the FARS1 gene, at the mRNA level, e.g., detecting a non-wild type level of a mRNA; or

detecting, in a tissue of the subject, the misexpression of the gene, at the protein level, e.g., detecting a non-wild type level of a FARS1 polypeptide.

In preferred embodiments the method includes: ascertaining the existence of at least one of: a deletion of one or more nucleotides from the FARS1 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:1, or naturally occurring mutants thereof or 5′ or 3′ flanking sequences naturally associated with the FARS1 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the FARS1 gene; the presence of a non-wild type splicing pattern of a messenger RNA transcript of the gene; or a non-wild type level of FARS1.

Methods of the invention can be used prenatally or to determine if a subject's offspring will be at risk for a disorder.

In preferred embodiments the method includes determining the structure of a FARS1 gene, an abnormal structure being indicative of risk for the disorder.

In preferred embodiments the method includes contacting a sample form the subject with an antibody to the FARS1 protein or a nucleic acid, which hybridizes specifically with the gene. There and other embodiments are discussed below.

Diagnostic and Prognostic Assays

The presence, level, or absence of FARS1 protein or nucleic acid in a biological sample can be evaluated by obtaining a biological sample from a test subject and contacting the biological sample with a compound or an agent capable of detecting FARS1 protein or nucleic acid (e.g., mRNA, genomic DNA) that encodes FARS1 protein such that the presence of FARS1 protein or nucleic acid is detected in the biological sample. The term “biological sample” includes tissues, cells and biological fluids isolated from a subject, as well as tissues, cells and fluids present within a subject. A preferred biological sample is serum. The level of expression of the FARS1 gene can be measured in a number of ways, including, but not limited to: measuring the mRNA encoded by the FARS1 genes; measuring the amount of protein encoded by the FARS1 genes; or measuring the activity of the protein encoded by the FARS1 genes.

The level of mRNA corresponding to the FARS1 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length FARS1 nucleic acid, such as the nucleic acid of SEQ ID NO:1, SEQ ID NO:6, or the DNA insert of the plasmid deposited with ATCC as Accession Number ______, or a portion thereof, such as an oligonucleotide of at least 7, 15, 30, 50, 100, 250 or 500 nucleotides in length and sufficient to specifically hybridize under stringent conditions to FARS1 mRNA or genomic DNA. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by the FARS1 genes.

The level of mRNA in a sample that is encoded by FARS1 can be evaluated with nucleic acid amplification, e.g., by rtPCR (U.S. Pat. No. 4,683,202), ligase chain reaction (Barany (1991) Proc. Natl. Acad. Sci. USA 88:189-193), self sustained sequence replication (Guatelli, et al., (1990) Proc. Natl. Acad. Sci. USA 87:1874-1878), transcriptional amplification system (Kwoh, et al. (1989) Proc. Natl. Acad. Sci. USA 86:1173-1177), Q-Beta replicase (Lizardi, et al. (1988) Bio/Technology 6:1197), rolling circle replication (U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the FARS1 gene being analyzed.

In another embodiment, the methods further contacting a control sample with a compound or agent capable of detecting FARS1 mRNA, or genomic DNA, and comparing the presence of FARS1 mRNA or genomic DNA in the control sample with the presence of FARS1 mRNA or genomic DNA in the test sample.

A variety of methods can be used to determine the level of protein encoded by FARS1. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect FARS1 protein in a biological sample in vitro as well as in vivo. In vitro techniques for detection of FARS1 protein include enzyme linked immunosorbent assays (ELISAs), immunoprecipitations, immunofluorescence, enzyme immunoassay (EIA), radioimmunoassay (RIA), and Western blot analysis. In vivo techniques for detection of FARS1 protein include introducing into a subject a labeled anti-FARS1 antibody. For example, the antibody can be labeled with a radioactive marker whose presence and location in a subject can be detected by standard imaging techniques.

In another embodiment, the methods further include contacting the control sample with a compound or agent capable of detecting FARS1 protein, and comparing the presence of FARS1 protein in the control sample with the presence of FARS1 protein in the test sample.

The invention also includes kits for detecting the presence of FARS1 in a biological sample. For example, the kit can include a compound or agent capable of detecting FARS1 protein or mRNA in a biological sample; and a standard. The compound or agent can be packaged in a suitable container. The kit can further comprise instructions for using the kit to detect FARS1 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of-control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted FARS1 expression or activity. As used herein, the term “unwanted” includes an unwanted phenomenon involved in a biological response such as pain or deregulated cell proliferation.

In one embodiment, a disease or disorder associated with aberrant or unwanted FARS1 expression or activity is identified. A test sample is obtained from a subject and FARS1 protein or nucleic acid (e.g., mRNA or genomic DNA) is evaluated, wherein the level, e.g., the presence or absence, of FARS1 protein or nucleic acid is diagnostic for a subject having or at risk of developing a disease or disorder associated with aberrant or unwanted FARS1 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted FARS1 expression or activity. For example, such methods can be used to determine whether a subject can be effectively treated with an agent for a CNS disorder, a metabolic disorder, and cell growth and differentiative disorder.

The methods of the invention can also be used to detect genetic alterations in a FARS1 gene, thereby determining if a subject with the altered gene is at risk for a disorder characterized by misregulation in FARS1 protein activity or nucleic acid expression, such as a CNS disorder, a metabolic disorder, and a cell growth and differentiative disorder. In preferred embodiments, the methods include detecting, in a sample from the subject, the presence or absence of a genetic alteration characterized by at least one of an alteration affecting the integrity of a gene encoding a FARS1-protein, or the mis-expression of the FARS1 gene. For example, such genetic alterations can be detected by ascertaining the existence of at least one of 1) a deletion of one or more nucleotides from a FARS1 gene; 2) an addition of one or more nucleotides to a FARS1 gene; 3) a substitution of one or more nucleotides of a FARS1 gene, 4) a chromosomal rearrangement of a FARS1 gene; 5) an alteration in the level of a messenger RNA transcript of a FARS1 gene, 6) aberrant modification of a FARS1 gene, such as of the methylation pattern of the genomic DNA, 7) the presence of a non-wild type splicing pattern of a messenger RNA transcript of a FARS1 gene, 8) a non-wild type level of a FARS1-protein, 9) allelic loss of a FARS1 gene, and 10) inappropriate post-translational modification of a FARS1-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the FARS1-gene. This method can include the steps of collecting a sample of cells from a subject, isolating nucleic acid (e.g., genomic, mRNA or both) from the sample, contacting the nucleic acid sample with one or more primers which specifically hybridize to a FARS1 gene under conditions such that hybridization and amplification of the FARS1-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

In another embodiment, mutations in a FARS1 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in FARS1 can be identified by hybridizing a sample and control nucleic acids, e.g., DNA or RNA, two dimensional arrays, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in FARS1 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the FARS1 gene and detect mutations by comparing the sequence of the sample FARS1 with the corresponding wild-type (control) sequence. Automated sequencing procedures can be utilized when performing the diagnostic assays ((1995) Biotechniques 19:448), including sequencing by mass spectrometry.

Other methods for detecting mutations in the FARS1 gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in FARS1 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in FARS1 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control FARS1 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci USA 86:6230).

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a FARS1 gene.

Use of FARS1 Molecules as Surrogate Markers

The FARS1 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the FARS1 molecules of the invention may be detected, and may be correlated with one or more biological states in vivo. For example, the FARS1 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The FARS1 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In-this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a FARS1 marker) transcription or expression, the amplified marker may be in a quantity which is more readily detectable than the drug itself. Also, the marker may be more easily detected due to the nature of the marker itself; for example, using the methods described herein, anti-FARS1 antibodies may be employed in an immune-based detection system for a FARS1 protein marker, or FARS1-specific radiolabeled probes may be used to detect a FARS1 mRNA marker. Furthermore, the use of a pharmacodynamic marker may offer mechanism-based prediction of risk due to drug treatment beyond the range of possible direct observations. Examples of the use of pharmacodynamic markers in the art include: U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am. J. Health-Syst. Pharm. 56 Suppl. 3: S16-S20.

The FARS1 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., FARS1 protein or RNA) for specific tumor markers in a subject, a drug or course of treatment may be selected that is optimized for the treatment of the specific tumor likely to be present in the subject. Similarly, the presence or absence of a specific sequence mutation in FARS1 DNA may correlate FARS1 drug response. The use of pharmacogenomic markers therefore permits the application of the most appropriate treatment for each subject without having to administer the therapy.

Pharmaceutical Compositions

The nucleic acid and polypeptides, fragments thereof, as well as anti-FARS1 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment

The present invention provides for both prophylactic and therapeutic methods of treating a subject at risk of (or susceptible to) a disorder or having a disorder associated with aberrant or unwanted FARS1 expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype,” or “drug response genotype”). Thus, another aspect of the invention provides methods for tailoring an individual's prophylactic or therapeutic treatment with either the FARS1 molecules of the present invention or FARS1 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted FARS1 expression or activity, by administering to the subject a FARS1 or an agent which modulates FARS1 expression or at least one FARS1 activity. Subjects at risk for a disease which is caused or contributed to by aberrant or unwanted FARS1 expression or activity can be identified by, for example, any or a combination of diagnostic or prognostic assays as described herein. Administration of a prophylactic agent can occur prior to the manifestation of symptoms characteristic of the FARS1 aberrance, such that a disease or disorder is prevented or, alternatively, delayed in its progression. Depending on the type of FARS1 aberrance, for example, a FARS1 agonist or FARS1 antagonist agent can be used for treating the subject. The appropriate agent can be determined based on screening assays described herein.

It is possible that some FARS1 disorders can be caused, at least in part, by an abnormal level of gene product, or by the presence of a gene product exhibiting abnormal activity. As such, the reduction in the level and/or activity of such gene products would bring about the amelioration of disorder symptoms. For example, in CNS disorders, metabolic disorders, and cell growth and differentiative disorders. The FARS1 molecules can also act as novel diagnostic targets and therapeutic agents for controlling one or more of cellular proliferative and/or differentiative disorders, disorders associated with aberrant or deficient lipid metabolism, energy metabolism, skeletal muscle function, cardiovascular function, CNS function, hepatic function, and renal function.

“Treatment”, as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, palliate, improve or affect the disease, the symptoms of disease or the predisposition toward disease. A therapeutic agent includes, but is not limited to, small molecules, peptides, antibodies, ribozymes and antisense oligonucleotides.

As discussed, successful treatment of FARS1 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of FARS1 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by FARS1 expression is through the use of aptamer molecules specific for FARS1 protein. Aptamers are nucleic acid molecules having a tertiary structure which permits them to specifically bind to protein ligands (see, e.g. Osborne, et al., (1997) Curr. Opin. Chem. Biol. 1: 5-9; and Patel, D. J. (1997) Curr. Opin. Chem. Biol. 1:32-46). Since nucleic acid molecules may in many cases be more conveniently introduced into target cells than therapeutic protein molecules may be, aptamers offer a method by which FARS1 protein activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of FARS1 disorders. For a description of antibodies, see the Antibody section above.

In circumstances wherein injection of an animal or a human subject with a FARS1 protein or epitope for stimulating antibody production is harmful to the subject, it is possible to generate an immune response against FARS1 through the use of anti-idiotypic antibodies (see, for example, Herlyn, D. Ann Med 1999;31(1):66-78; and Bhattacharya-Chatterjee, M., and Foon, K. A. Cancer Treat Res 1998;94:51-68). If an anti-idiotypic antibody is introduced into a mammal or human subject, it should stimulate the production of anti-anti-idiotypic antibodies, which should be specific to the FARS1 protein. Vaccines directed to a disease characterized by FARS1 expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993, Proc. Natl. Acad. Sci. USA 90:7889-7893).

The identified compounds that inhibit target gene expression, synthesis and/or activity can be administered to a patient at therapeutically-effective doses to prevent, treat or ameliorate FARS1 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate FARS1 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al (1996) Current Opinion in Biotechnology 7:89-94 and in Shea, K. J. (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al (1993) Nature 361:645-647. Through the use of isotope-labeling, the “free” concentration of compound which modulates the expression or activity of FARS1 can be readily monitored and used in calculations of IC50.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC50. An rudimentary example of such a “biosensor” is discussed in Kriz, D. et al (1995) Analytical Chemistry 67:2142-2144.

Another aspect of the invention pertains to methods of modulating FARS1 expression or activity for therapeutic purposes. Accordingly, in an exemplary embodiment, the modulatory method of the invention involves contacting a cell with a FARS1 or agent that modulates one or more of the activities of FARS1 protein activity associated with the cell. An agent that modulates FARS1 protein activity can be an agent as described herein, such as a nucleic acid or a protein, a naturally-occurring target molecule of a FARS1 protein (e.g., a FARS1 substrate or receptor), a FARS1 antibody, a FARS1 agonist or antagonist, a peptidomimetic of a FARS1 agonist or antagonist, or other small molecule.

In one embodiment, the agent stimulates one or FARS1 activities. Examples of such stimulatory agents include active FARS1 protein and a nucleic acid molecule encoding FARS1. In another embodiment, the agent inhibits one or more FARS1 activities. Examples of such inhibitory agents include antisense FARS1 nucleic acid molecules, antiFARS1 antibodies, and FARS1 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a FARS1 protein or nucleic acid molecule. In one embodiment, the method involves administering an agent (e.g., an agent identified by a screening assay described herein), or combination of agents that modulates (e.g., up regulates or down regulates) FARS1 expression or activity. In another embodiment, the method involves administering a FARS1 protein or nucleic acid molecule as therapy to compensate-for reduced, aberrant, or unwanted FARS1 expression or activity.

Stimulation of FARS1 activity is desirable in situations in which FARS1 is abnormally downregulated and/or in which increased FARS1 activity is likely to have a beneficial effect. For example, stimulation of FARS1 activity is desirable in situations in which a FARS1 is downregulated and/or in which increased FARS1 activity is likely to have a beneficial effect. Likewise, inhibition of FARS1 activity is desirable in situations in which FARS1 is abnormally upregulated and/or in which decreased FARS1 activity is likely to have a beneficial effect.

Pharmacogenomics

The FARS1 molecules of the present invention, as well as agents, or modulators which have a stimulatory or inhibitory effect on FARS1 activity (e.g., FARS1 gene expression) as identified by a screening assay described herein can be administered to individuals to treat (prophylactically or therapeutically) FARS1 associated disorders (e.g., CNS disorders, metabolic disorders, and cell growth and differentiative disorders) associated with aberrant or unwanted FARS1 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a FARS1 molecule or FARS1 modulator as well as tailoring the dosage and/or therapeutic regimen of treatment with a FARS1 molecule or FARS1 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11) :983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a FARS1 protein of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a FARS1 molecule or FARS1 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a FARS1 molecule or FARS1 modulator, such as a modulator identified by one of the exemplary screening assays described herein.

The present invention further provides methods for identifying new agents, or combinations, that are based on identifying agents that modulate the activity of one or more of the gene products encoded by one or more of the FARS1 genes of the present invention, wherein these products may be associated with resistance of the cells to a therapeutic agent. Specifically, the activity of the proteins encoded by the FARS1 genes of the present invention can be used as a basis for identifying agents for overcoming agent resistance. By blocking the activity of one or more of the resistance proteins, target cells, e.g., human cells, will become sensitive to treatment with an agent that the unmodified target cells were resistant to.

Monitoring the influence of agents (e.g., drugs) on the expression or activity of a FARS1 protein can be applied in clinical trials. For example, the effectiveness of an agent determined by a screening assay as described herein to increase FARS1 gene expression, protein levels, or upregulate FARS1 activity, can be monitored in clinical trials of subjects exhibiting decreased FARS1 gene expression, protein levels, or downregulated FARS1 activity. Alternatively, the effectiveness of an agent determined by a screening assay to decrease FARS1 gene expression, protein levels, or downregulate FARS1 activity, can be monitored in clinical trials of subjects exhibiting increased FARS1 gene expression, protein levels, or upregulated FARS1 activity. In such clinical trials, the expression or activity of a FARS1 gene, and preferably, other genes that have been implicated in, for example, a FARS1-associated disorder can be used as a “read out” or markers of the phenotype of a particular cell.

Other Embodiments

In another aspect, the invention features, a method of analyzing a plurality of capture probes. The method can be used, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence; contacting the array with a FARS1, preferably purified, nucleic acid, preferably purified, polypeptide, preferably purified, or antibody, and thereby evaluating the plurality of capture probes. Binding, e.g., in the case of a nucleic acid, hybridization with a capture probe at an address of the plurality, is detected, e.g., by signal generated from a label attached to the FARS1 nucleic acid, polypeptide, or antibody.

The capture probes can be a set of nucleic acids from a selected sample, e.g., a sample of nucleic acids derived from a control or non-stimulated tissue or cell.

The method can include contacting the FARS1 nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of FARS1. Such methods can be used to diagnose a subject, e.g., to evaluate risk for a disease or disorder, to evaluate suitability of a selected treatment for a subject, to evaluate whether a subject has a disease or disorder. FARS1 is associated with CNS disorders, metabolic disorders, and cell growth and differentiative disorders, thus it is useful for evaluating the same.

The method can be used to detect SNPs, as described above.

In another aspect, the invention features, a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express FARS1 or from a cell or subject in which a FARS1 mediated response has been elicited, e.g., by contact of the cell with FARS1 nucleic acid or protein, or administration to the cell or subject FARS1 nucleic acid or protein; providing a two dimensional array having a plurality of addresses, each address of the plurality being positionally distinguishable from each other address of the plurality, and each address of the plurality having a unique capture probe, e.g., wherein the capture probes are from a cell or subject which does not express FARS1 (or does not express as highly as in the case of the FARS1 positive plurality of capture probes) or from a cell or subject-which in which a FARS1 mediated response has not been elicited (or has been elicited to a lesser extent than in the first sample); contacting the array with one or more inquiry probes (which is preferably other than a FARS1 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features, a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or mis express FARS1 or from a cell or subject in which a FARS1 mediated response has been elicited, e.g., by contact of the cell with FARS1 nucleic acid or protein, or administration to the cell or subject FARS1 nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express FARS1 (or does not express as highly as in the case of the FARS1 positive plurality of capture probes) or from a cell or subject which in which a FARS1 mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features, a method of analyzing FARS1, e.g., analyzing structure, function, or relatedness to other nucleic acid or amino acid sequences. The method includes: providing a FARS1 nucleic acid or amino acid sequence; comparing the FARS1 sequence with one or more preferably a plurality of sequences from a collection of sequences, e.g., a nucleic acid or protein sequence database; to thereby analyze FARS1.

The method can include evaluating the sequence identity between a FARS1 sequence and a database sequence. The method can be performed by accessing the database at a second site, e.g., over the internet.

In another aspect, the invention features, a set of oligonucleotides, useful, e.g., for identifying SNP's, or identifying specific alleles of FARS1. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with differential labels, such that an oligonucleotides which hybridizes to one allele provides a signal that is distinguishable from an oligonucleotides which hybridizes to a second allele.

The sequence of a FARS1 molecules is provided in a variety of mediums to facilitate use thereof. A sequence can be provided as a manufacture, other than an isolated nucleic acid or amino acid molecule, which contains a FARS1. Such a manufacture can provide a nucleotide or amino acid sequence, e.g., an open reading frame, in a form which allows examination of the manufacture using means not directly applicable to examining the nucleotide or amino acid sequences, or a subset thereof, as they exists in nature or in purified form.

A FARS1 nucleotide or amino acid sequence can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a FARS1 sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features, a method of analyzing a sequence. The method includes: providing a FARS1 sequence, or record, in computer readable form; comparing a second sequence to the FARS1 sequence; thereby analyzing a sequence. Comparison can include comparing to sequences for sequence identity or determining if one sequence is included within the other, e.g., determining if the FARS1 sequence includes a sequence being compared. In a preferred embodiment the FARS1 or second sequence is stored on a first computer, e.g., at a first site and the comparison is performed, read, or recorded on a second computer, e.g., at a second site. E.g., the FARS1 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

This invention is further illustrated by the following examples which should not be construed as limiting. The contents of all references, patents and published patent applications cited throughout this application are incorporated herein by reference.

### EXAMPLES

### Example 1

### Identification and Characterization of FARS1 cDNA

Mouse FARS1 (mFARS1) was identified in a search for secreted proteins in a library from mouse epididymal white fat. An oligo-dT primed library was constructed in the vector pMET7 using standard methods, and random clones from this library were sequenced. The mFARS1 clone contains an open reading frame of 822 nucleotides flanked by 397 and 1673 nucleotides at the 5′ and 3′ ends, respectively. MFARS1 is predicted to encode a 273 amino acid secreted protein with a signal sequence at the N-terminus of 21 amino acids (amino acids 1-21 of SEQ ID NO:7, shown in SEQ ID NO:9) and no other obvious membrane spanning domains. The FARS1 protein sequence did not show similarity to other proteins in the database. Notable are the presence of 6 cysteines in the mFARS1 sequence (at positions 11, 22, 35, 83, 109, and 186 of SEQ ID NO:7) which is predicted to be involved in folding of the processed protein. Also notable are six potential dibasic cleavage sites (at positions R151-R152; K201-K202; K212-K213; K222-K223; K225-R226; and R232-K233 of SEQ ID NO:7) which suggests a further processing of FARS1 into peptides.

A partial clone of human FARS1 was identified in a proprietary database by homology searching. The 5′ end of the human clone was identified among genomic sequences in the public database by homology searching. The DNA sequence of the assembled clone with the translation product is shown in SEQ ID NO:1.

An alignment of the coding region of mFARS1 and hFARS1 nucleotide sequence indicates that these two sequences share 86% identity. The human and murine FARS1 polypeptide sequences are 87% identical. An alignment of the two polypeptide sequences indicates that both the six cysteines and all of the potential dibasic cleavage sites are conserved between mouse and human FARS1 sequences. The sequence similarity is distributed unevenly throughout the protein: the C-terminal 40 amino acids (corresponding to one predicted peptide) are only 65% identical between human and mouse FARS1, whereas the sequence encompassing another processed peptide, e.g., amino acids 153 to 200, are more than 90% identical to each other.

### Example 2

### Tissue Distribution of FARS1 mRNA

Tissues were collected from 7 week old female C57/B16J mice and 6 week old male C57/B16J mice. Total RNA was prepared using the trizol method and treated with DNAse to remove contaminating genomic DNA. cDNA was synthesized using standard techniques. Mock cDNA synthesis in the absence of reverse transcriptase resulted in samples with no detectable PCR amplification of the control 18S RNA gene confirming effiecient removal of genomic DNA contamination. FARS1 expression was measured by TaqMan® quantitative PCR analysis, performed according to the manufacturer's directions (Perkin Elmer Applied Biosystems, Foster City, Calif.).

The tissue samples included the following normal mouse tissues: BAT (brown adipose tissue), WAT (white adipose tissue), brain-hypothalamus, hypothalamus, skeletal muscle, liver, kidney, heart, intestine, and spleen.

PCR probes were designed by PrimerExpress software (PE Biosystems) based on the sequence of murine FARS1. The following probes and primers were used:

| | |
|---|---|
| mFARS1 forward primer: | |
| 5′ AATGGTGGCCCTCCAGATCT 3′ | (SEQ ID NO:11) |
| mFARS1 reverse primer: | |
| 5′ GGTCAATGGTGTAGAGCTGAAGG 3′ | (SEQ ID NO:12) |
| mFARS1 probe: | |
| 5′ CCGTGCCACACTGTTCCACCAATAGAAA 3′ | (SEQ ID NO:13) |

To standardize the results between different tissues, two probes, distinguished by different fluorescent labels, were added to each sample. The differential labeling of the probe for the FARS1 gene and the probe for 18S RNA as an internal control thus enabled their simultaneous measurement in the same well. Forward and reverse primers and the probes for both 18S RNA and human or murine 14273 were added to the TaqMan Universal PCR Master Mix (PE Applied Biosystems). Although the final concentration of primer and probe could vary, each was internally consistent within a given experiment. A typical experiment contained 200 nM of forward and reverse primers, plus 100 nM of the probe for the 18S RNA, and 600 nM of each of the forward and reverse primers, plus 200 nM of the probe for mFARS1. TaqMan matrix experiments were carried out using an ABI PRISM 770 Sequence Detection System (PE Applied Biosystems). The thermal cycler conditions were as follows: hold for 2 minutes at 50° C. and 10 minutes at 95° C., followed by two-step PCR for 40 cycles of 95° C. for 15 seconds, followed by 60° C. for 1 minute.

The following method was used to quantitatively calculate mFARS1 gene expression in the tissue samples, relative to the 18S RNA expression in the same tissue. The threshold values at which the PCR amplification started were determined using the manufacturer's software. PCR cycle number at threshold value was designated as CT. Relative expression was calculated as 2^{-((CTtest-CT18S)tissue of interest-(CTtest-CT18S)lowest expressing tissue in panel)}. Samples were run in duplicate and the averages of 2 relative expression determinations are shown. All probes were tested on serial dilutions of RNA from a tissue with high expression levels and only probes which gave relative expression levels that were linear to the amount of template cDNA with a slope similar to the slope for the internal control 18S were used.

FARS1 expression in mouse tissues was also measured by Northern blot analysis, performed with ³²P-labeled DNA probes using the rapid-HYB buffer (Amersham). The tissues examined included BAT, WAT, pancreas, kidney, heart, brain, spleen, lung, liver, skeletal muscle, and smooth muscle.

The results of TaqMan analysis of tissue expression of mFARS1 showed that the highest levels of expression were in brown adipose tissue, white adipose tissue, skeletal muscle, and hypothalamus. Northern blot analysis confirmed the high levels of mFARS1 expression in white and brown adipose tissue, brain, and skeletal muscle. See Table 1.

TABLE 1

| | Relative FARS1 |
|---|---|
| Tissue | Experssion |
| BAT | 107 |
| WAT | 41 |
| Brain- | 13.5 |
| Hypothalamus | |
| Hypothalamus | 34.6 |
| Skeletal | 57.9 |
| Muscle | |
| Liver | 12.8 |
| Kidney | 15.4 |
| Heart | 28.3 |
| Intestine | 2.07 |
| Spleen | 1.06 |

To determine whether mFARS1 expression is associated with a change in body weight, mFARS1 was measured in tissues of mice that had been slowly dieted to 80% or overfed to 120% of normal body weight. As determined by TaqMan analysis, mFARS1mRNA in epididymal white fat was increased 1.7-fold in the overfed mice and decreased 2.5-fold in the underfed mice, as compared to control animals. See Table 2.

TABLE 2

| | Relative FARS1 |
|---|---|
| Diet | Expression |
| WAT control | 1.01 |
| WAT underfed | 0.42 |
| WAT overfed | 1.22 |

### Example 3

### Recombinant Expression of FARS1 in Bacterial Cells

In this example, FARS1 is expressed as a recombinant glutathione-S-transferase (GST) fusion polypeptide in E. coli, and the fusion polypeptide is isolated and characterized. Specifically, FARS1 is fused to GST and this fusion polypeptide is expressed in E. coli, e.g., strain PEB199. Expression of the GST-FARS1 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 4

### Expression of Recombinant FARS1 Protein in COS Cells

To express the FARS1 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire FARS1 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the FARS1 DNA sequence is amplified by PCR using two primers. The 5′ primer contains the restriction site of interest followed by approximately twenty nucleotides of the FARS1 coding sequence starting from the initiation codon; the 3′ end sequence contains complementary sequences to the other restriction site of interest, a translation stop codon, the HA tag or FLAG tag and the last 20 nucleotides of the FARS1 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the FARS1 gene is inserted in the correct orientation. The ligation mixture is transformed into E. coli cells (strains HB101, DH5I, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the FARS1-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the FARS1 polypeptide is detected by radiolabelling (35S-methionine or ³⁵S-cysteine available from NEN, Boston, Mass., can be used) and immunoprecipitation (Harlow, E. and Lane, D. (1988) Antibodies: A Laboratory Manual, Cold Spring Harbor Laboratory Press, Cold Spring Harbor, N.Y.,) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the FARS1 coding sequence is cloned directly into the polylinker of the pcDNA/Amp vector using the appropriate restriction sites. The resulting plasmid is transfected into COS cells in the manner described above, and the expression of the FARS1 polypeptide is detected by radiolabelling and immunoprecipitation using a FARS1 specific monoclonal antibody.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule comprising a nucleotide sequence which is at least 80% identical to the nucleotide sequence of SEQ ID NO:1, SEQ ID NO:3, SEQ ID NO:6, or SEQ ID NO:8;
b) a nucleic acid molecule comprising a fragment of the nucleotide sequence of SEQ ID NO:1, SEQ ID NO:3, SEQ ID NO:6, or SEQ ID NO:8;
c) a nucleic acid molecule which encodes a polypeptide comprising the amino acid sequence of SEQ ID NO:2, SEQ ID NO:5, SEQ ID NO:7, or SEQ ID NO:10, or a biologically active fragment thereof;
d) a nucleic acid molecule which encodes a naturally occurring allelic variant of a polypeptide comprising the amino acid sequence of SEQ ID NO:2, SEQ ID NO:5, SEQ ID NO:7, or SEQ ID NO:10; and
e) a nucleic acid molecule which encodes a naturally occurring allelic variant of a polypeptide comprising the amino acid sequence of SEQ ID NO:2, SEQ ID NO:5, SEQ ID NO:7, or SEQ ID NO:10, wherein the nucleic acid molecule hybridizes to a nucleic acid molecule comprising SEQ ID NO:1, SEQ ID NO:3, SEQ ID NO:6, SEQ ID NO:8, or a complement thereof, under stringent conditions.

2. The isolated nucleic acid molecule of claim 1, which is selected from the group consisting of:
a) a nucleic acid molecule comprising the nucleotide sequence of SEQ ID NO:1, SEQ ID NO:3, SEQ ID NO:6, or SEQ ID NO:8;
b) a nucleic acid molecule which encodes a polypeptide comprising the amino acid sequence of SEQ ID NO:2, SEQ ID NO:5, SEQ ID NO:7, or SEQ ID NO:10; and
c) a naturally occurring allelic variant of a nucleic acid molecule comprising the nucleotide sequence shown in SEQ ID NO:1, SEQ ID NO:3, SEQ ID NO:6, or SEQ ID NO:8, wherein the nucleic acid molecule hybridizes under stringent hybridization conditions to a nucleic acid molecule comprising the nucleotide sequence of SEQ ID NO:1, SEQ ID NO:3, SEQ ID NO:6, or SEQ ID NO:8.

3. The nucleic acid molecule of claim 1 further comprising vector nucleic acid sequences.

4. A host cell which contains the nucleic acid molecule of claim 1.

5. The host cell of claim 5 which is a mammalian host cell.

6. The host cell of claim 6 which is a non-human mammalian host cell.

7. The nucleic acid molecule of claim 1 further comprising nucleic acid sequences encoding a heterologous polypeptide.

8. An isolated polypeptide selected from the goup consisting of:
a) a polypeptide which is encoded by a nucleic acid molecule comprising a nucleotide sequence which is at least 80% identical to a nucleic acid comprising the nucleotide sequence of SEQ ID NO:1, SEQ ID NO:3, SEQ ID NO:6, or SEQ ID NO:8;
b) a polypeptide comprising an amino acid sequence which is at least 80% identical to the amino acid sequence shown in SEQ ID NO:2, SEQ ID NO:5, SEQ ID NO:7, or SEQ ID NO:10;
c) a naturally occurring variant of a polypeptide comprising the amino acid sequence shown in SEQ ID NO:2, SEQ ID NO:5, SEQ ID NO:7, or SEQ ID NO:10;
d) a polypeptide which is encoded by a nucleic acid molecule comprising a nucleotide sequence which hybridizes under stringent hybridization conditions to a complement of a nucleic acid molecule comprising the nucleotide sequence of SEQ ID NO:1, SEQ ID NO:3, SEQ ID NO:6, or SEQ ID NO:8; and
e) a fragment of a polypeptide comprising the amino acid sequence of SEQ ID NO:2, SEQ ID NO:5, SEQ ID NO:7, or SEQ ID NO:10, wherein the fragment comprises at least 15 contiguous amino acids of SEQ ID NO:2, SEQ ID NO:5, SEQ ID NO:7, or SEQ ID NO:10.

9. The isolated polypeptide of claim 8 comprising the amino acid sequence of SEQ ID NO:2, SEQ ID NO:5, SEQ ID NO:7, or SEQ ID NO:10.

10. The polypeptide of claim 8 further comprising one or more heterologous amino acid sequences.

11. An antibody which selectively binds to a polypeptide of claim 8.

12. A method for producing a polypeptide, wherein the method comprises introducing into a host cell a nucleic acid molecule encoding a polypeptide sequence selected from the group consisting of:
a) a polypeptide comprising the amino acid sequence of SEQ ID NO:2, SEQ ID NO:5, SEQ ID NO:7, or SEQ ID NO:10;
b) a polypeptide comprising a fragment of the amino acid sequence of SEQ ID NO:2, SEQ ID NO:5, SEQ ID NO:7, or SEQ ID NO:10, wherein the fragment comprises at least 15 contiguous amino acids of SEQ ID NO:2, SEQ ID NO:5, SEQ ID NO:7, or SEQ ID NO:10; and
c) a naturally occurring variant of the FARS1 polypeptide which is encoded by a nucleic acid molecule which hybridizes under stringent conditions to a complement of a nucleic acid molecule comprising SEQ ID NO:1, SEQ ID NO:3, SEQ ID NO:6, or SEQ ID NO:8.

13. A method for detecting the presence of a polypeptide of claim 8 in a sample, comprising:
a) contacting the sample with a compound which selectively binds to a polypeptide of claim 8; and
b) determining whether the compound binds to the polypeptide in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds to a polypeptide of claim 8 comprising the steps of:
a) contacting a polypeptide, or a cell expressing a polypeptide of claim 8 with a test compound; and
b) determining whether the polypeptide binds to the test compound.

20. The method of claim 19, wherein the binding of the test compound to the polypeptide is detected by a method selected from the group consisting of:
a) detection of binding by direct detecting of test compound/polypeptide binding;
b) detection of binding using a competition binding assay; and
c) detection of binding using an assay for FARS1-mediated signal transduction.

21. A method for modulating the activity of a polypeptide of claim 8 comprising contacting a polypeptide or a cell expressing a polypeptide of claim 8 with a compound which binds to the polypeptide in a sufficient concentration to modulate the activity of the polypeptide.

22. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.
